(19) United States
(12) Patent Application Publication
(10) Pub. No.: US 2011/0040232 A1
(43) Pub. Date: Feb. 17, 2011

(54) DUODENAL LINER DEVICE

(75) Inventor: Elad Magal, Ramat Hasharon (IL)

Correspondence Address: GREENBERG TRAURIG, LLP, 200 PARK AVE., P.O. BOX 677, FLORHAM PARK, NJ, 07932, US

(73) Assignee: Duo-Cure, Inc.

(21) Appl. No.: 12/988,971

(22) PCT Filed: Jan. 22, 2009
(86) PCT No.: PCT/IB09/50253

§ 371 Date: Oct. 21, 2010

Related U.S. Application Data
(60) Provisional application No. 61/047,299, filed on Apr. 23, 2008.
(60) Provisional application No. 61/122,514, filed on Dec. 15, 2008.

(51) Int. Cl. A61F 2/04

(52) U.S. Cl. 604/8; 606/192

(57) ABSTRACT

Disclosed are tethered duodenal liner devices as well as methods of transcutaneously deploying and removing the tethered duodenal liner devices.

### RELATED APPLICATION

The present application is gains priority from U.S. Provisional Patent Applications No. 61/047,299 filed 23 Apr. 2008 and 61/122,514 filed 15 Dec. 2008, both which are included by reference as if fully set forth herein.

### FIELD AND BACKGROUND OF THE INVENTION

The present invention, in some embodiments thereof, relates to the treatment of conditions related to eating disorders such as obesity and overeating. Some embodiments of the invention relate to methods and devices related to duodenal liner devices useful for providing a beneficial effect for treating conditions relating to eating disorders.

The gastrointestinal tract of placental mammals such as humans is a tube passing from the mouth to the anus, having various physically different regions, each having different functions and corresponding structures. Food passing through the lumen of the gastrointestinal tract is processed at the different regions to be digested, allowing nutrients and energy to be absorbed while expelling waste.

The upper portion of a gastrointestinal tract 10 of a human, schematically depicted in cross section in FIG. 1 as viewed from the front, includes an esophagus 12, a stomach 14 and a duodenum 16.

Stomach 14 is a hollow J-shaped organ having muscular gastric walls defining a gastric cavity 18. A lower esophageal sphincter 20 opens into the large cardiac portion of stomach 14 defined between a lesser curvature 22 on the right and a greater curvature 24 to the left. Above the cardiac portion is fundus 26. Below the cardiac portion, is a pyloric portion 28 of stomach 14, including a pyloric antrum 30 and terminating with a pyloric sphincter 32.

Pylorus 34, the distal aperture of stomach 14, is defined by pyloric sphincter 32 and leads to the lumen of duodenum 16. Pylorus 34 is ordinarily tightly closed by pyloric sphincter 32 to prevent reflux from duodenum 16 to the stomach 14.

Duodenum 16, the most proximal part of the small intestine, is approximately 24 cm long. In an adult the course of the duodenum describes an almost 270° imperfect circle divided into four roughly linear portions: the first (superior) portion; the second (descending) portion; the third (transverse) portion; and the fourth (ascending) portion.

The superior portion of duodenum 16 is about 5 cm long commencing at pyloric sphincter 32 and passing backwards, upwards, and rightwards to the neck of the gall-bladder, varying slightly in direction according to the degree of distension of the stomach. Unlike the other portions of duodenum 16, pyloric sphincter 32 and pyloric portion 28 of stomach 14, the superior portion of duodenum 16 is relatively immotile. The part of the superior portion of duodenum 16 that abuts pyloric sphincter 32 has a larger diameter than other portions of duodenum 16 defining a slightly bulging cavity termed a duodenal bulb 36.

In a stomach 14, ingested food is liquefied into chyme by the contractions of the gastric walls to churn the food in the presence of hydrochloric acid and digestive enzymes. When sufficiently processed in stomach 14, the chyme is expelled through pylorus 34 into the duodenum 16. In duodenum 16, the acidic chyme is neutralized and digested by bile and enzymes.

Obesity is a result, a symptom and/or a cause of many pathological conditions. One concept for the treatment of obesity is the reduction of the amount of energy absorbed from an ingested volume of food. In one implementation of this concept, the efficiency of digestion is reduced making less of the energy of the food available for absorption. An accepted method for both reducing the efficiency of digestion of ingested food is by deploying a gastrointestinal liner in the gastrointestinal tract.

Gastrointestinal liners include a liner tube having walls of material defining a liner lumen deployed inside a portion of the gastrointestinal tract to function as an intraluminal gastrointestinal bypass device. In some cases, duodenal liners, gastrointestinal liners deployed at least partially in the duodenum are considered to act analogously to, and provide many of the advantages of a Roux-en-Y gastric bypass, including weight loss and control of type-2 diabetes.

A typical duodenal liner device is described in U.S. Pat. No. 7,267,694 where the proximal end of a flexible, floppy liner tube of impermeable material defining a liner lumen is endoscopically deployed and anchored with the help of a barbed stent in the pylorus or in the superior section of the duodenum, the stent also ensuring that the proximal lumen opening the liner tube remains open. Chyme from the stomach enters the open proximal lumen opening of the liner tube and passes through the liner lumen to the distal lumen opening. Digestive enzymes secreted in the duodenum pass through the duodenum on the outside of the liner tube. The enzymes and the chyme do not mix until the chyme exits from the distal lumen opening of the liner tube. In such a way, the efficiency of the process of digestion of the chyme is diminished, reducing the ability of the gastrointestinal tract to absorb calories from the food.

G.I. Dynamics, Inc., (Watertown, Mass., USA) produces the Endobarrier® device that is substantially a duodenal liner device configured so that the proximal end of the device is anchored inside the duodenal bulb with the help of a barbed anchoring stent that also keeps the proximal lumen opening open.

In US 2004/0148034 is taught a duodenal liner device attached to a funnel, the funnel configured for anchored to the gastric walls inside the gastric cavity in proximity to the lower esophageal sphincter. Food passing the lower esophageal sphincter is directed by the funnel into the proximal lumen opening of the duodenal liner device.

In U.S. Pat. No. 7,121,283 is taught a duodenal liner device attached to a large stent-like anchoring device that presses outwardly against the pyloric portion of the stomach, the pyloric sphincter and the duodenal bulb.

### SUMMARY OF THE INVENTION

Some embodiments of the invention relate to duodenal liners having advantages over known duodenal liners. Specifically, some embodiments of the invention relate to duodenal liners secured in place, inter alia, with the help of a tether.

According to an aspect of some embodiments of the invention, there is provided a duodenal liner device, comprising:
a) a liner tube configured for deployment inside a duodenum of a human subject, the liner tube having walls of a flexible material defining a liner lumen, a proximal end defining a proximal lumen opening, and a distal end defining a distal lumen opening;
b) an expandable centering component functionally associated with the proximal end of the liner tube, having a collapsed configuration and an expanded deployed configuration; and
c) an elongated tether having a proximal tether end and a distal tether end, the distal tether end functionally associated with the proximal end of the liner tube, the tether configured to pass through a gastric wall of a subject in which deployed.

In some embodiments, the centering component is configured to maintain the proximal lumen opening of the liner tube substantially centered with a duodenal lumen when deployed in a duodenum in the deployed configuration.

In some embodiments, the centering component is configured to maintain the proximal lumen opening of the liner tube dilated when in the deployed configuration.

In some embodiments, the centering component is configured to assist in preventing excessive distal migration of the liner tube into a gastrointestinal tract when deployed in a duodenum in the deployed configuration.

In some embodiments, the walls of the liner tube are substantially impermeable.

In some embodiments, the centering component has an axial length not greater than a radius in the deployed configuration.

In some embodiments, the centering component has an axial length not less than a radius in the deployed configuration.

In some embodiments, the centering component is integrally formed with the liner tube.

In some embodiments, the centering component is secured to the liner tube.

In some embodiments, the centering component is expandable, from the collapsed configuration to the deployed configuration.

In some embodiments, the centering component is reversibly expandable, from the deployed configuration to the collapsed configuration.

In some embodiments, the centering component is self-expanding.

In some embodiments, the centering component is expandable by application of an outwards radial force to an inner surface of the centering component.

In some embodiments, the centering component comprises a balloon. In such embodiments a balloon is of any suitable shaped, e.g., barrel-shaped, cylindrical, ovoid, toroidal, ring-shaped, partially ring shaped, and spiral.

In some embodiments, the centering component comprises a stent. In some embodiments the stent is a self-expanding stent. In some embodiments, the stent is expandable by application of an outwards radial force to an inner surface of the stent.

In some embodiments, the distal tether end contacts the centering component so as to be functionally associated with the liner tube. In some embodiments, the distal tether end is secured to the centering component.

In some embodiments, the distal tether end contacts the liner tube so as to be functionally associated with the liner tube. In some embodiments, the distal tether end is secured to the liner tube.

In some embodiments, the tether has an outer diameter of not more than about 5 mm, not more than about 3 mm, not more than about 2 mm and even not more than about 1 mm.

In some embodiments, the tether is filamentous, including a wire, string, thread, ribbon or yarn. In some embodiments the tether is a single filament. In some embodiments the tether is fashioned from multiple filaments. In some embodiments the tether is coated.

In some embodiments, the tether comprises a tube with an internal lumen.

In some embodiments, the internal lumen of the tether constitutes a conduit for transport of a fluid to controllably change a configuration of the centering component. In some embodiments, the controllable changing comprises changing the centering component from a collapsed configuration to a deployed configuration. In some embodiments, the controllable changing comprises changing an outer dimension of the centering component when deployed.

In some embodiments, the duodenal liner device further comprises an anchor configured to engage the proximal end of the tether. In some embodiments, an anchor comprises a pad for contacting tissue.

In some embodiments, an anchor is configured for subcutaneous implantation.

In some embodiments, the tether configured to pass through the skin of a subject in which deployed. In some embodiments, an anchor is configured for extracutaneous deployment.

In some embodiments, the duodenal liner device further comprises a composition-administration component, configured to facilitate administration of a composition to a gastrointestinal tract in which deployed. In some embodiments, the composition-administration component configured to administer a fluid composition as a liquid. In some embodiments, the composition-administration component is configured to administer a fluid composition as a spray. In some embodiments, the composition-administration component is configured to administer a composition in the superior portion of the duodenum. In some embodiments, the composition-administration component is configured to administer a composition in the ascending portion of the duodenum.

In some embodiments, the tether comprises a tube with an internal lumen, the internal lumen constituting a conduit for transport of a composition to the composition-administration component.

In some embodiments, a portion of a surface of a component of the duodenal liner configured for deployment in a duodenal bulb is substantially entirely smooth.

In some embodiments, a portion of a surface of a component of the duodenal liner configured for deployment in a duodenal bulb comprises features for increasing resistance of that portion to movement in a distal direction from the duodenal bulb. In some embodiments, the features are configured to provide little resistance to movement in a proximal direction from the duodenal bulb. In some embodiments, at least some of the features are integrally formed with the portion. In some embodiments, at least some of the features are distally directed protrusions. In some embodiments, the features are located on an outer surface of the liner tube. In some embodiments, the features are located on an outer surface of the centering component in an expanded state.

In some embodiments, the device further comprises a gastric balloon. In some embodiments, the gastric balloon is physically linked to the tether.

According to an aspect of some embodiments of the invention, there is also provided a deployment device for deploying a duodenal liner device in the gastrointestinal tract of a human, comprising:
a) a duodenal liner device including:
   i. a liner tube having a wall of flexible material defining a liner lumen, a proximal end defining a proximal lumen opening and a distal end defining a distal lumen opening; and
   ii. an expandable centering component functionally associated with a proximal end of the wall of the liner tube, having a collapsed configuration and an expanded deployed configuration;
b) an elongated probe section having a distal opening, containing at least a portion of the centering component of the duodenal liner device.

In some embodiments, the duodenal liner device is a duodenal liner device described above.

In some embodiments, the probe section is substantially rigid. In some embodiments, the probe section is substantially tubular. In some embodiments, the probe section is configured to pass through an abdominal wall.

In some embodiments, the deployment device further comprises a pusher configured to slide inside the probe section to push the centering component out of the probe section through the distal end of the probe section.

In some embodiments, the deployment device is sterilized and packaged in a sterility-preserving package.

According to an aspect of some embodiments of the invention, there is also provided a method for deploying a duodenal liner device in the gastrointestinal tract of a human subject, comprising:
a) establishing a transcutaneous channel from outside of the body of the subject through a gastric wall;
b) transporting a duodenal liner device through the channel, past the gastric wall, into a gastric cavity and through a pylorus of the subject;
c) deploying an expandable centering component of the duodenal liner device in a duodenal bulb of the subject, the centering component functionally associated with a liner tube of flexible material defining a liner lumen of the duodenal liner device;
d) deploying the liner tube of flexible material in the duodenum;
e) functionally associating a distal end of an elongated tether to a proximal end of the liner tube;
f) passing a proximal end of the tether from the proximal end of the liner tube through a gastric wall of the subject and into the transcutaneous channel; and
g) substantially fixing the position of the proximal end of the tether relative to the duodenal bulb of the subject.

In some embodiments, the transcutaneous channel is substantially straight.

In some embodiments, the transcutaneous channel enters the body of the subject from the left side of the body of the subject.

In some embodiments, the transcutaneous channel enters the body of the subject at a point located no higher than the top of the twelfth thoracic vertebra T12 and no lower than the bottom of the second lumbar vertebra L2.

In some embodiments, the transcutaneous channel penetrates the gastric wall at the greater curvature of a stomach of the subject.

In some embodiments, the transcutaneous channel penetrates the gastric wall near a pyloric portion of a stomach of the subject.

In some embodiments, the tether passes through the gastric cavity substantially in parallel to a pyloric antrum of a stomach of the subject.

In some embodiments, during the transporting of the duodenal liner device, at least part of the duodenal liner device is contained within a probe section of a deployment device. In some embodiments, the probe section of the deploying device is substantially tubular and part of the duodenal liner device is contained inside the lumen of the probe section. In some embodiments, the probe section of the deploying device is substantially rigid.

In some embodiments, the transcutaneous channel is formed using the probe section.

In some embodiments, the probe passes through a cannula, the cannula maintaining the channel dilated.

In some embodiments, the deploying of the centering component comprises expanding the centering component from a collapsed configuration to a deployed configuration. In some embodiments, subsequent to the deploying of the centering component, a portion of the centering component presses against walls of the duodenal bulb of the subject.

In some embodiments, subsequent to the deploying of the centering component, a portion of the centering component lightly contacts walls of the duodenal bulb of the subject.

In some embodiments, the centering component is self-expanding, during the transporting of the duodenal liner device, the centering component is constrained to the collapsed configuration, and the deploying of the centering component comprises releasing the centering component from the constraints, allowing the centering component to adopt the deployed configuration.

In some embodiments, the centering component is a balloon, during the transporting of the duodenal liner device the centering component is in a collapsed configuration; and the deploying of the centering component comprises forcing fluid into the balloon, so that the centering component adopts the deployed configuration.

In some embodiments, the centering component is expandable by application of an outwards radial force, during the transporting of the duodenal liner device the centering component is in a collapsed configuration; and the deploying of the centering component comprises applying an outwards radial force to the centering component so that the centering component adopts the deployed configuration.

In some embodiments, the deploying of the liner tube of flexible material comprises directing fluid into the liner lumen.

In some embodiments, the deploying of the liner tube of flexible material comprises pushing a solid liner tube-deployer into the liner lumen.

In some embodiments, the tether is functionally associated with the proximal end of the liner tube prior to the transporting of the duodenal liner device through the channel.

In some embodiments, the tether is functionally associated with the proximal end of the liner tube subsequent to the deploying of the centering component.

In some embodiments, passing the proximal end of the tether into the transcutaneous channel is affected by transporting the distal end of the tether through the transcutaneous channel (in some embodiments, when the distal end of the tether is functionally associated with other components of the duodenal liner device) while the proximal end of the tether is maintained at the proximal end of the transcutaneous channel.

In some embodiments, passing the proximal end of the tether into the transcutaneous channel is affected by transporting the distal end and the proximal end of the tether through the transcutaneous channel (in some embodiments, when the distal end of the tether is functionally associated with other components of the duodenal liner device), and subsequently withdrawing the proximal end of the tether through the transcutaneous channel.

In some embodiments, the fixing of the proximal end of the tether comprises securing the proximal end of the tether to a subcutaneous anchor. In some embodiments, the subcutaneous anchor is configured to stay in place by the intermittent application of pressure to abdominal muscles.

In some embodiments, the fixing of the proximal end of the tether comprises securing the proximal end of the tether to an extracutaneous anchor. In some embodiments, the extracutaneous anchor is configured to stay in place by the intermittent application of pressure to skin.

According to an aspect of some embodiments of the invention, there is also provided a method for removing a duodenal liner device deployed in a duodenum of a human subject and at least partially secured in place with an elongated tether, the tether having a proximal end and a distal end functionally associated with the duodenal liner device, the method comprising:
a) exposing the proximal end of the tether;
b) guiding a probe section of an extraction device through a channel defined by the tether in the body of the subject to proximity of a proximal end of the duodenal liner device;
c) placing at least a portion of the duodenal liner device inside the probe section of the extraction device; and
d) extracting the probe section of the extraction device from the body of the subject through the channel
   thereby removing the duodenal liner device.

In some embodiments, exposing the proximal end of the tether comprises detaching the tether from an extracutaneous anchor.

In some embodiments, the distal end of the tether is subcutaneous and the exposing the proximal end of the tether comprises cutting skin. In some embodiments, exposing the proximal end of the tether comprises detaching the tether from a subcutaneous anchor.

In some embodiments, the probe section of the extraction device is substantially tubular. In some embodiments, the probe section of the extraction device is substantially axially rigid.

In some embodiments, the method further comprises increasing the radial diameter of the channel defined by the tether. In some embodiments, the increasing of the radial diameter of the channel is prior to the guiding of the probe section of the extraction device through the channel.

In some embodiments, the increasing of the radial diameter of the channel is during the guiding of the probe section of the extraction device through the channel.

In some embodiments, placing of a portion of the duodenal liner device inside the probe section of the extraction device comprises pulling the tether (in some embodiments while holding the extraction device substantially in place) in a proximal direction so as to pull the portion of the duodenal liner device into the probe section of the extraction device.

In some embodiments, placing of a portion of the duodenal liner device inside the probe section of the extraction device comprises pushing the extraction device (in some embodiments while holding the tether substantially in place) in a distal direction so as to push the probe section of the extraction device over the portion of the duodenal liner device.

In some embodiments, the method further comprises collapsing a centering component functionally associated with the proximal end of the duodenal liner device prior to the placing of the portion of the duodenal liner device in the probe section of the extraction device.

In some embodiments, the collapsing of the centering component comprises removing a fluid from the centering component.

In some embodiments, the collapsing of the centering component is a result of a force applied to the centering component by the probe section of the extraction device during the placing of the portion of the duodenal liner device in the probe section.

In some embodiments, the collapsing of the centering component comprises pulling a drawstring configured to collapse the centering component. In some embodiments, the drawstring is a component of the duodenal liner device. In some embodiments, the drawstring is a component of the extraction device.

Unless otherwise defined, all medical, technical and scientific terms used herein have the same meaning as commonly understood by one of ordinary skill in the art to which this invention pertains. In case of conflict, the patent specification, including definitions, will control.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the described composition, device or method.

As used herein, the indefinite articles “a” and “an” mean “at least one” or “one or more” unless the context clearly dictates otherwise.

Herein the term “proximal” generally refers to the side or end of an elongated medical device such as a catheter that is intended to be closer to the performing medical personnel, further from the location of the intervention and is generally located outside the body of the patient. Herein the term “proximal” also refers to the side or end of an elongated organ such as a duodenum that that is closer to the mouth.

Herein the term “distal” generally refers to the side or end of an elongated medical device such as a catheter that is intended to be closer to or at the location of the intervention, for example the duodenum. The term “distal” also refers to the side or end of an elongated organ such as a duodenum that is further from the mouth.

Herein, the term “liner tube” and “sleeve” may, depending on the context, be used interchangeably, as accepted in common in the art.

### BRIEF DESCRIPTION OF THE FIGURES

Some embodiments of the invention are herein described, by way of example only, with reference to the accompanying figures. The description, together with the figures, makes apparent how embodiments of the invention may be practiced to a person having ordinary skill in the art. The figures are for the purpose of illustrative discussion of embodiments of the invention and no attempt is made to show structural details of an embodiment in more detail than is necessary for a fundamental understanding of the invention. For the sake of clarity, some objects depicted in the figures are not to scale.

In the Figures:

FIG. 1 (prior art) is a schematic depiction of part of a human gastrointestinal tract including the stomach and duodenum, in cross section view from the front;

FIGS. 2A-2C schematically depict an embodiment of a duodenal liner device of the invention;

FIG. 3A schematically depicts an embodiment of the device of FIG. 2 packed inside a deployment device;

FIGS. 3B-3F schematically depict various stages in the deployment of the device of FIG. 2 in a duodenum;

FIGS. 4A-4B schematically depict various stages in the removal of the device of FIG. 2 from a duodenum;

FIGS. 5A-5F schematically depict an embodiment of a duodenal liner device of the invention;

FIG. 6 schematically depicts and embodiment of a duodenal liner device of the invention;

FIGS. 7A-7D schematically depict an embodiment of a duodenal liner device of the invention; and

FIGS. 8A-8G schematically depict an embodiment of a duodenal liner device of the invention.

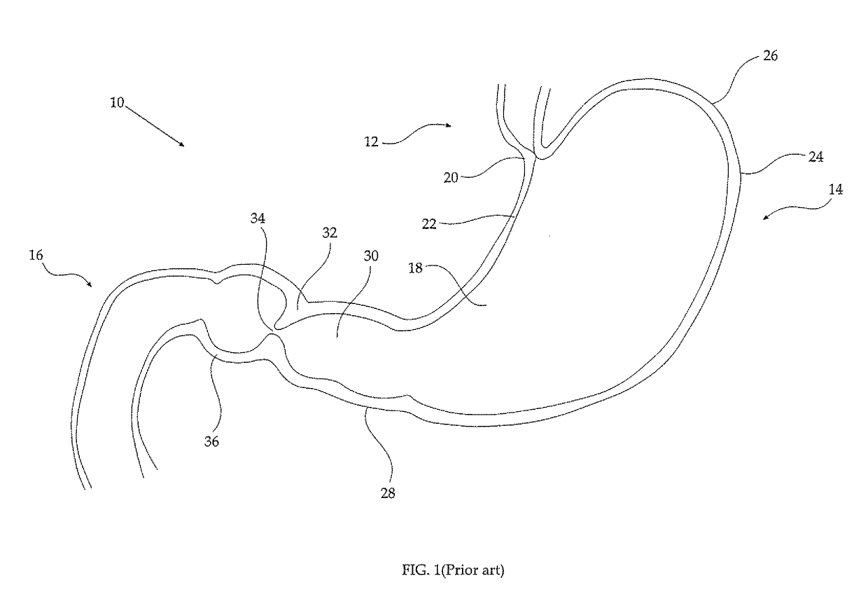

### DESCRIPTION OF SOME EMBODIMENTS OF THE INVENTION

Some embodiments of the invention relate to methods and devices related to duodenal liner devices useful for providing a beneficial effect for treating conditions relating to eating disorders. Specifically, some embodiments of the present invention relate to duodenal liner devices that are secured in place, inter alia, with the help of a tether.

Some embodiments of the present invention have at least one beneficial effect. Beneficial effects include effects such as curing a condition, treating a condition, preventing a condition, treating symptoms of a condition, curing symptoms of a condition, ameliorating symptoms of a condition, treating effects of a condition, ameliorating effects of a condition, and preventing results of a condition. For example, in some embodiments the beneficial effects are similar to or the same as beneficial effects of prior art duodenal liner devices including reducing the amount of energy absorbed from an ingested amount of food and thus have a beneficial effect for persons suffering from overweight and obesity or control of Type II diabetes.

The principles, uses and implementations of the teachings of the invention may be better understood with reference to the accompanying description and figures. Upon perusal of the description and figures present herein, one skilled in the art is able to implement the teachings of the invention without undue effort or experimentation. In the figures, like reference numerals refer to like parts throughout.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

As noted above, a duodenal liner device generally includes a liner tube having walls of material defining a liner lumen. A duodenal liner device is deployed inside at least part of the duodenum so that the liner tube constitutes an intraluminal bypass of at least part of the duodenum.

One of the primary challenges of duodenal liners is that of anchoring. If a duodenal liner is insufficiently anchored, intestinal peristaltic forces pull the liner tube distally into the small intestine leading to bowel obstruction requiring urgent surgical intervention.

The Endobarrier® as well as duodenal liner devices taught in U.S. Pat. No. 7,267,694 (both of G.I. Dynamics, Inc., Watertown, Mass., USA) include a barbed stent secured to the proximal end of the liner tube to anchor the liner tube in place and to keep the proximal end of the liner tube open. During deployment, the stent is expanded inside the duodenal bulb. To ensure sufficient anchoring, the stent is expanded to the extent that the stent presses into the tissue and applies constant pressure to the walls of the duodenal bulb. The constant pressure applied to the duodenal bulb desensitizes duodenal mechanoreceptors and may change the shape of the duodenal bulb. The scarring around the barbs as well as tissue growth over and around the stent changes the nature of the surface of the duodenal bulb, changing the functioning of the duodenal bulb including of sensory nerves and glands necessary for normal functioning of the duodenum. The tissue growth around the stent and the barbs make removal of the duodenal liner device challenging and even dangerous.

Some embodiments of the present invention relate to the anchoring of a duodenal liner with the help of a tether that passes through a gastric wall of a subject when deployed. In some embodiments, the tether defines and passes through a channel in the body of the subject. In some embodiments, the channel is substantially straight.

In some embodiments, the tether passes from the duodenum through the pylorus, pyloric antrum and into the pyloric portion of the stomach of a subject in which deployed. In some embodiments, the tether passes into the gastric wall near a pyloric portion of the stomach of the subject. In some embodiments, the tether passes into the gastric wall at the greater curvature of the stomach of the subject. In some embodiments, the channel enters the body from the left side of the body of the subject. In to some embodiments, the channel is transcutaneous and enters the body at a point located no higher than the top of the twelfth thoracic vertebra T12 and no lower than the bottom of the second lumbar vertebra L2.

### Duodenal Liner Device

The teachings of the invention are preferably implemented with a duodenal liner device of the invention. In some embodiments, a duodenal liner device comprises:

a) a liner tube configured for deployment inside a duodenum of a human subject, the liner tube having walls of a flexible material defining a liner lumen, a proximal end defining a proximal lumen opening, and a distal end defining a distal lumen opening;

b) an expandable centering component functionally associated with the proximal end of the liner tube, having a collapsed configuration and an expanded deployed configuration; and

c) an elongated tether having a proximal tether end and a distal tether end, the distal tether end functionally associated with the proximal end of the liner tube, the tether configured to pass through a gastric wall of a subject in which deployed.

### Liner Tube

As noted above, some embodiments of the invention include a liner tube of a material defining a liner lumen, a proximal end defining a proximal lumen opening and a distal end defining a distal lumen opening. In some embodiments, the proximal end of the liner tube is functionally associated with an expandable centering component. Like liner tubes known in the art, a liner tube used in implementing embodiments of the invention is configured for deployment inside a duodenum of a mammal, such as a human.

Generally, the liner tube itself is sufficiently flexible to follow the curvature of the duodenum. Further, in some embodiments the walls of the liner tube are sufficiently flexible and/or collapsible to allow duodenal peristalsis to drive chyme through the lumen of the liner tube. Sufficient collapsibility of the walls of the liner tube prevents continuous intimate contact of the outer surface of the liner tube with the duodenal mucosa, avoiding damage to the duodenal mucosa and allowing digestive secretions as well as chyme not collected into the liner lumen to pass through the duodenal lumen outside the liner lumen.

In some embodiments, at least a portion of the wall of a liner tube is porous or semipermeable to allow entry of digestive secretions into the liner lumen and/or to allow the flow of fluids and digested matter out of the liner lumen.

In some embodiments, at least a portion of the wall of a liner tube is impermeable (analogous to the Endobarrier® by GI Dynamics Inc, Watertown, Mass., USA and as described in U.S. Pat. No. 7,267,694 which is included by reference as if fully set forth herein).

In some embodiments, the diameter of the liner lumen is substantially constant along the entire length of the liner tube. Although any suitable luminal diameter may be used, in some embodiments, the luminal diameter is not more than about 30 mm, not more than about 25 mm and even not more than about 20 mm. That said, the proximal end of the liner tube of material is of a size to match the centering component, in embodiments having a smooth and continuous transition from a wider diameter near the centering component to a smaller distal diameter. Consequently, in some embodiments, the proximal end of the liner tube is flared and in some embodiments, the centering component and the proximal end of the liner tube together define a funnel-like structure.

The length of the liner tube is any suitable length and is in embodiments selected in accordance with clinical decisions made by the treating physician. That said, a typical liner tube is between about 25 cm and about 160 cm long. Generally, the liner tube is selected so that when the duodenal liner device is deployed, the distal lumen opening of the liner tube is located distal to the duodenal-jejunal flexure and empties out into the jejunum. In some embodiments, the liner tube is even longer.

Suitable materials from which embodiments of a liner tube for implementing the invention are fashioned include silicone, polyurethane, polyethylene (e.g., low density polyethylene films) and fluoropolymers (e.g., expanded polytetrafluoroethylene). In some embodiments, a liner tube is fashioned from fluoropolymer or polyethylene film impregnated with polyurethane or silicone to reduce permeability, as taught in U.S. Pat. No. 7,267,694.

In some embodiments, a liner tube comprises an anti-buckling or anti-twisting component to reduce the chance of buckling and/or twisting, as taught in U.S. Pat. No. 7,267,694.

In some embodiments, some or the entire liner tube is configured to be visible using a medical imaging modality, for example is made of a material or includes features that are detectable with the help of an imaging modality, for example include radio-opaque portions detectable with X-ray imaging modalities or sono-opaque portions detectable with ultrasonic imaging modalities. In some embodiments, markers visible in an imaging modality are arranged along the length of the liner tube, for example in a line, to allow medial personnel to determine if the liner tube is twisted. If the liner tube is twisted, untwisting can be performed as described in U.S. Pat. No. 7,267,694.

A liner tube used in implementing the invention may be or may resemble a liner tubel used in implementing duodenal liner devices known in the art. The type and properties of a liner tube used is dependent on medical criteria including the condition treated, the severity of the condition and decisions made by the treating physician. A person having ordinary skill in the art is familiar with various duodenal liner devices, for example duodenal liner devices described in US 2004/148034 and U.S. Pat. No. 7,267,694.

### Elongated Tether

In some embodiments, a duodenal liner device of the invention comprises a tether, which is substantially an elongated, flexible component part of which function is to prevent release of a deployed device into the gastrointestinal tract with concomitant gastrointestinal blockage and the associated catastrophic results. When a duodenal liner device is deployed, a distal end of the tether is functionally associated with the proximal end of the liner tube while the proximal end passes through at least the gastric wall and is anchored in place. In some embodiments the distal end of the tether contacts the liner tube and in some embodiments is directly secured to the liner tube. In some embodiments the distal end of the tether contacts and even secured to a different component of the duodenal liner device, for example the centering component, and is therefore functionally associated to the proximal end of the liner tube indirectly.

In a preferred embodiment, a tether is sufficiently strong not to break under the pulling force applied by peristalsis to the liner tube. Preferred tethers are sutures, wires, tubes and like components. In preferred embodiment, the outer surface of the tether is substantially impervious to gastric juices and is tissue-friendly, being non-allergenic and discouraging microbial growth. In some embodiments, a tether is configured to avoid sticking to tissue so that the tether can slide past organs and through tissue without damaging the tissue or organs.

In typical embodiments, the outer surface of a tether is of a material such as used in the manufacture of percutaneous endoscopic gastrostomy tubes, such as polyethylene, polypropylene, polyethylene terephthalate (Dacron®), fluorinated hydrocarbons (e.g., polytetrafluoroethylene), silicone, polyvinylchloride, latex, polyurethane, silicone polyurethane copolymers, synthetic polyisoprene and other materials.

In some embodiments, a tether comprises one or more strands of one or more materials, e.g., polyethylene (e.g., UHMWPE such as Dyneema® (Koninklijk DSM, Heerlen, The Netherlands) or Spectra® (Honeywell, Morris Township, N.J., USA)). Such materials are usually sufficiently strong, tissue friendly and substantially impervious to gastric juices.

In some embodiments, a tether is a coated, for example is a stainless steel wire coated with or encased inside PTFE. In some such embodiments, the wire provides the required strength while the coating or encasing layer is tissue-friendly, preventing cutting and other tissue trauma.

In some embodiments, a tether is a hollow liner tube and comprises one or more lumina. In some such embodiments, the tether is configured to function as a fluid conduit, as detailed hereinbelow.

It is generally preferred that the outer diameter of a tether be as small as possible to be as unobtrusive as possible, to cause as little tissue trauma as possible and to avoid substantial leakage of gastric fluids. Thus, although tethers of any suitable size may be used, in some embodiments the outer diameter of a tether is not more than about 5 mm, not more than about 3 mm, not more than about 2 mm and even not more than about 1 mm.

In some embodiments, a distal end of a tether of a duodenal liner device of the invention contacts or is secured to the centering component of the device, in some embodiments to the proximal end of the centering component. In some embodiments, a distal end of a tether contacts or is secured to the liner tube.

In some embodiments, the distal end of the tether is secured at more than one location, for example of the centering component or of the liner tube. For example, in some embodiments, especially where the centering component is prone to orienting perpendicularly to the duodenal axis when deployed (e.g., is relatively short), the distal end of the tether is secured at at least three points, for example of the centering component or of the liner tube so that the tether stabilizes the position of the centering component.

### Expandable Centering Component

In some embodiments, a centering component of a duodenal liner device of the invention is functionally associated with the proximal end of the liner tube.

In some embodiments, when the duodenal liner device is deployed and the centering component is in a deployed configuration, the centering component maintains the proximal lumen opening of the liner tube substantially centered with the duodenal lumen and the pylorus, so that chyme passing the pylorus enters the proximal lumen opening of the liner tube.

In some embodiments, when the duodenal liner device is deployed and the centering component is in a deployed configuration, the centering component helps maintain the proximal lumen opening of the liner tube dilated so that chyme passing the pylorus enters the proximal lumen opening of the liner tube.

In some embodiments, when the duodenal liner device is deployed and the centering component is in a deployed configuration, the centering component acts as an anchoring component, helping to preventing excessive distal migration of the liner tube into the gastrointestinal tract.

Generally, a centering component has at least two configurations: a collapsed (smaller dimension) configuration for deployment and an expanded (larger dimension) deployed configuration where the centering component.

It is generally preferred that in a collapsed configuration the centering component be as small as possible, allowing deployment with minimal trauma.

In some embodiments, the centering component has an axial length not greater than a radius in the deployed configuration. In some embodiments, the centering component has an axial length not less than a radius in the deployed configuration.

In some embodiments, a centering component is reversibly-expandable, allowing a deployed centering component to be collapsed to a collapsed configuration for simple maintenance, removal and/or repositioning.

In some embodiments, a centering component is self-expanding, that is to say, has an inherent tendency to adopt a deployed configuration when free of constraints. In some such embodiments, the self-expanding force is relatively weak so that when deployed inside a gastrointestinal tract, the force applied by the centering component does not press into and damage the gastrointestinal intima. In some embodiments, the size of the centering component in the expanded state is similar to or somewhat smaller than that of the region of the gastrointestinal tract in which deployed, e.g., the duodenal bulb.

In some embodiments, a centering component is controllably expandable, that is to say, is configured to be controllably changed from a collapsed configuration to a deployed configuration. Such configuration allows the size of the centering component in the deployed configuration to be selected during deployment.

### Anchor

In some embodiments, when a duodenal liner device of the invention is deployed, a proximal end of a tether of the device passes through at least the gastric wall and is anchored in place. Generally an anchor is a solid object. In some embodiments, an anchor comprises a pad that distributes pulling forces applied by the tether on a surface. A person having ordinary skill in the art is acquainted with suitable anchors, for example anchors similar to (although in some embodiments different in shape and/or dimensions) to anchors used in implementing the Coapsys® device commercially available from Myocor, Inc. (Maple Grove, Minn., USA).

In some embodiments, the tether passes only through the gastric wall and is secured to an anchor located on and contacting an outer surface of the stomach, in some embodiments the peritoneum. In some such embodiments, an anchor distributes forces over the outer surface of the stomach.

In some embodiments, the tether passes through the gastric wall as well as other tissue to an anchor that contacts a muscle layer, for example an abdominal muscle layer. In some such embodiments, an anchor distributes forces over the outer surface of the muscle layer. An advantage of some such embodiments is that the anchor and proximal tip of the tether are hidden under the skin, reducing the chance of infection and providing a more esthetic appearance, yet are readily accessible by cutting through the skin.

In some embodiments, the tether passes through the gastric wall as well as other tissue to emerge through the skin, to be secured to an anchor that contacts the outer skin surface. A person having ordinary skill in the art is acquainted with suitable such anchors, for examples extracutaneous anchors used with commercially available PEG devices, e.g., a MIC™ feeding tube (Ballard Medical Products, Draper, Utah, USA).

### Passage Through Layers of Tissue

In some embodiments, when a duodenal liner device of the invention is deployed, a tether of the device passes through a gastric wall and, in some embodiments, other layers of tissues.

In some embodiments, a tether passes directly through layers of tissue, that is to say, the outer surface of the tether contacts the tissue. In such embodiments, the tether may be considered as defining a channel through the body of the subject in which deployed.

In some embodiments, a tether does not pass directly through layers of tissue, but rather through a passage defined by an implanted channel-defining component. For example, in some embodiments a PEG device (e.g., a commercially-available PEG device such as a MIC™ gastrostomy feeding tube (Ballard Medical Products, Draper, Utah, USA) defines a channel through the body of the subject through which a tether passes. In some such embodiments, the tether does not necessarily contact tissue during passage in the channel, but rather contacts portions of the passage defined by of the channel-defining component. Generally, the luminal size of the passage defined by the channel-defining component is close to the external diameter of the tether to reduce or eliminate the chance of leakage.

In some such embodiments, the tether is functionally associated with a dedicated anchor that is not a component of the channel-defining component. In some such embodiments, the tether is functionally associated with an anchor that is a component of the channel-defining component, such as the external anchoring button of a PEG device.

### Composition Administration

In some embodiments, a duodenal liner device of the invention is configured to facilitate administration of a composition, e.g., a pharmaceutical composition to a gastrointestinal tract in which deployed.

In some such embodiments, a duodenal liner device comprises a composition-administration component for administration of a composition in or to the gastrointestinal tract. In some embodiments, the composition-administration component is physically associated with the liner tube or the centering component of the duodenal liner device. In some embodiments, the composition-administration component is configured to administer a composition in the superior portion of the duodenum. In some embodiments, a composition-administration component configured to administer a the composition in the ascending portion of the duodenum.

In some embodiments, a composition administration component comprises a transport conduit to transport composition from a reservoir to be administered. In some embodiments, the tether comprises a tube with an internal lumen, the internal lumen constituting a conduit for transport of a composition to the composition-administration component.

In some embodiments, the composition-administration component configured to administer a fluid composition as a liquid. In some such embodiments, the composition-administration component is configured in accordance with the teachings of PCT Patent Publication WO 2008/104968 of the Applicant (which is included by reference as if fully set forth herein) to administer a composition as a spray to the gastrointestinal tract, especially to the luminal surface of the duodenum.

For example, in some embodiments, the proximal section (e.g., in a section configured to be deployed within about 5 cm of the pylorus) of a liner tube of material and/or of a centering component of a duodenal liner device is fenestrated, allowing spraying of a composition therethrough against the luminal wall of the duodenum. For example, in some embodiments, on the outside of the proximal section (e.g., in a section configured to be deployed within about 5 cm of the pylorus) of a liner tube and/or of the centering component are situated sprayers, allowing administration of a desired composition against the luminal wall of the duodenum.

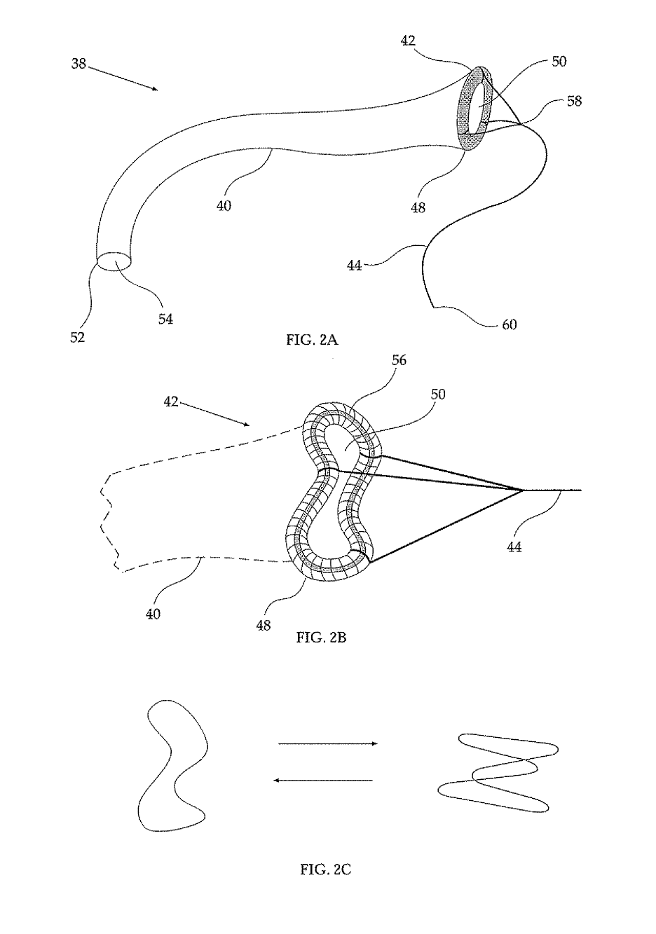

An embodiment of a duodenal liner of the invention, duodenal liner device 38 is depicted in FIGS. 2A, 2B and 2C. Duodenal liner device 38 comprises a liner tube 40, an expandable centering component 42 and a tether 44, see FIG. 2A.

Liner tube 40 is configured for deployment inside a duodenum of a human and includes walls of a flexible material defining a liner lumen, a proximal end 48 defining a proximal lumen opening 50, and a distal end 52 defining a distal lumen opening 54. The walls of liner tube 40 are impermeable, thin, flexible, collapsible and made of a fluoropolymer. Liner tube 40 is similar to the liner tube of the Endobarrier® by G.I. Dynamics, Inc., Watertown, Mass., USA.

Expandable centering component 42 is substantially a 1 mm diameter wire loop 56 of shape-memory Nitinol around which proximal end 48 of liner tube 40 is rolled, see FIG. 2B where liner tube 40 is drawn transparent. Wire loop 56 has a three-lobed saddle-shaped and has a deployed configuration in which wire loop 56 maintains proximal lumen opening 50 dilated. In FIG. 2C, wire loop 56 is depicted alone in a collapsed configuration and in deployed configuration.

Tether 44 is filamentous, substantially a 1 mm diameter strand of polyethylene (Dyneema®, DSM, Heerlen, The Netherlands). Distal end 58 of tether 44 opens to three separate parts that encircle wire loop 56 and the rolled sections of liner tube 40 at three separate points around the perimeter of centering component 42. In an expanded configuration, centering component 42 has an axial length not greater than a radius in the deployed configuration and therefore has a tendency to “tip over” when deployed in a duodenum. Association with tether 44 at three separate points around the perimeter of centering component 42 maintains centering component 42 perpendicular to the axis of the duodenum when deployed. Proximal end 60 of tether 44 is a loose strand that is smooth, impervious to gastric conditions, non-allergenic and has an extremely low coefficient of friction so is therefore suitable for passing through living tissue such as a gastric wall.

In some embodiments, a duodenal liner device of the invention is deployed using the method of the invention for deploying a duodenal finer in the gastrointestinal tract of a human subject.

### Method of Deploying a Duodenal Liner Device

An aspect of the present invention relates to a method for deploying a duodenal liner in the gastrointestinal tract of a human subject. In some embodiments, the method of the present invention includes producing a transcutaneous channel from outside of the body of the subject, through a gastric wall, past the gastric wall, into the gastric cavity and then deploying a centering component of the duodenal liner device in the duodenal bulb of the subject.

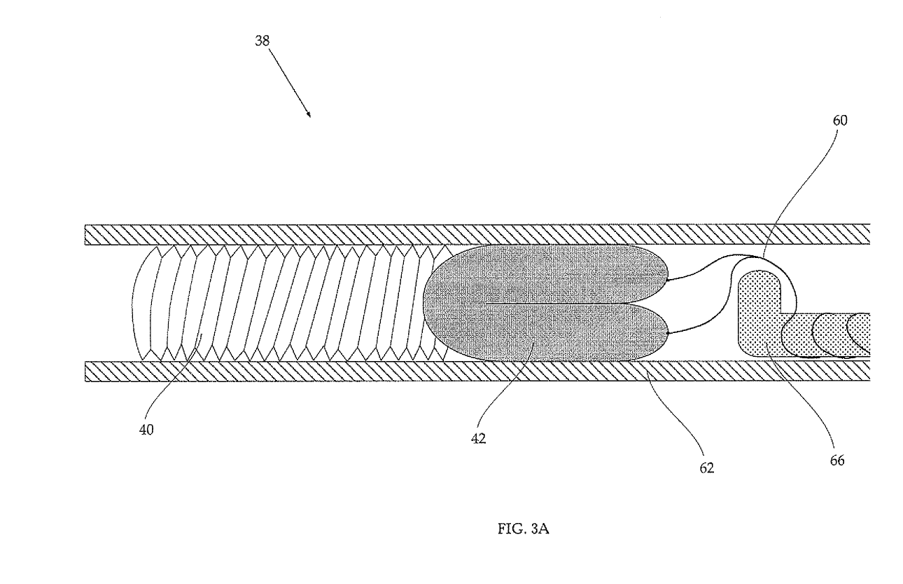

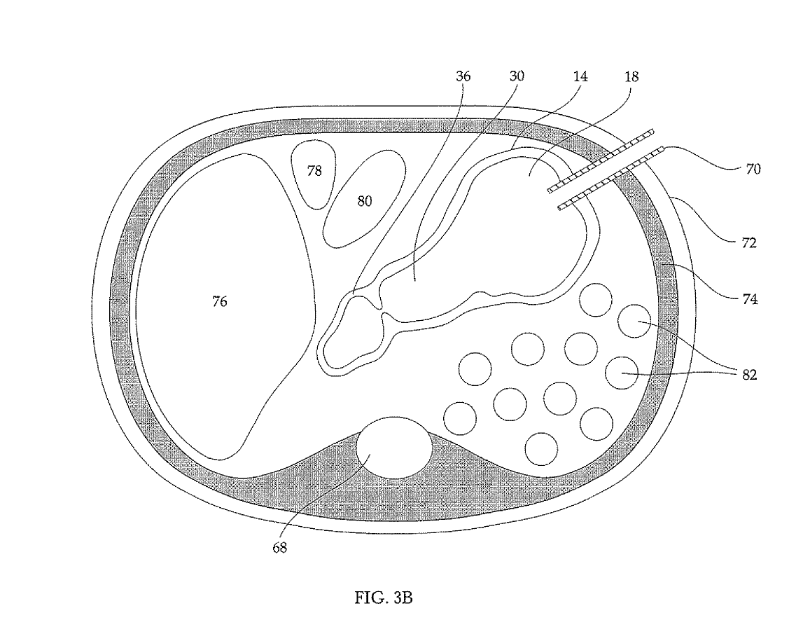

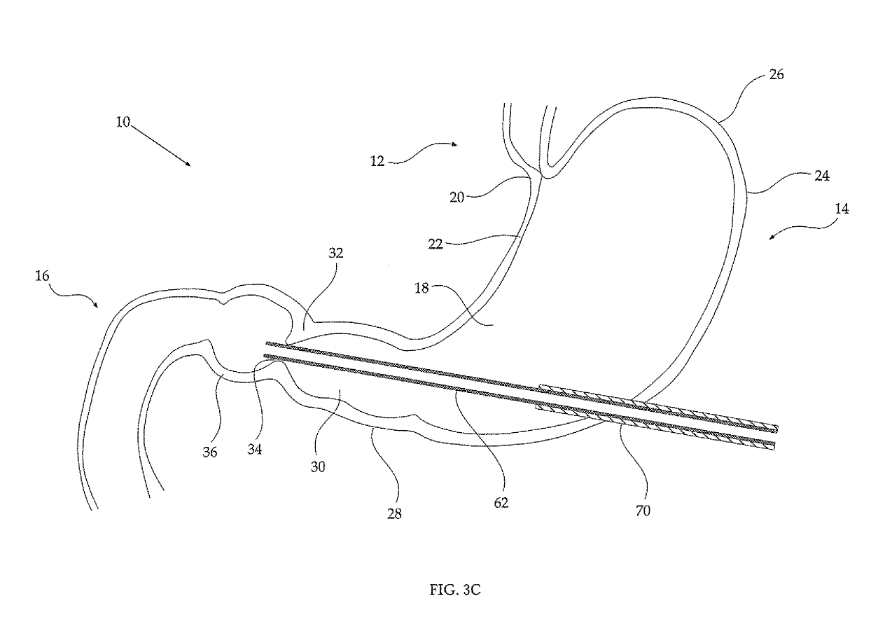

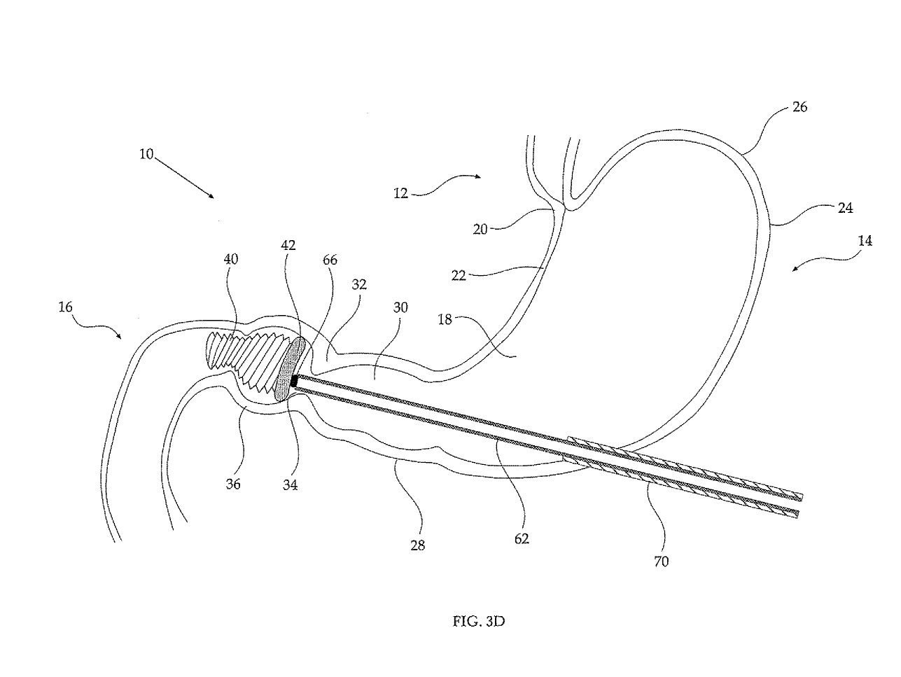

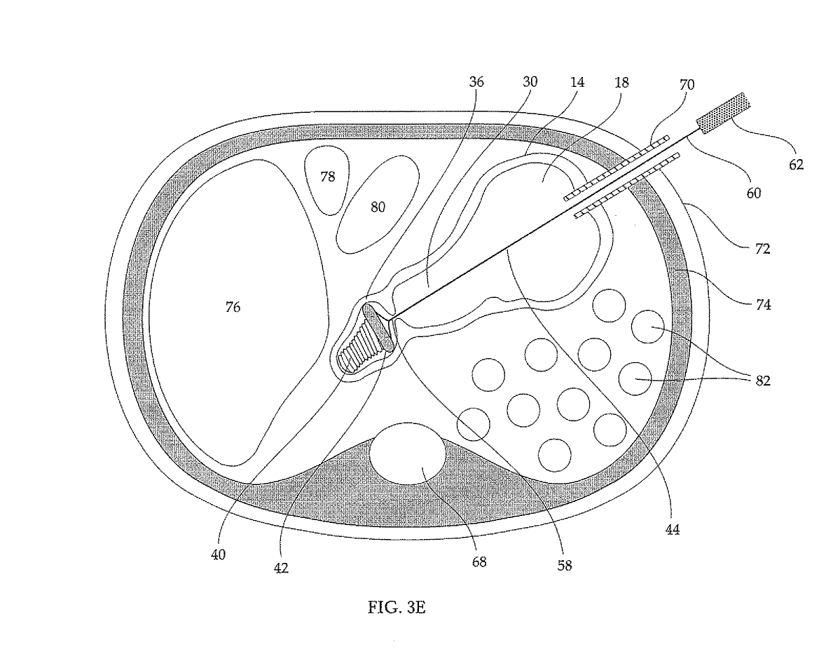

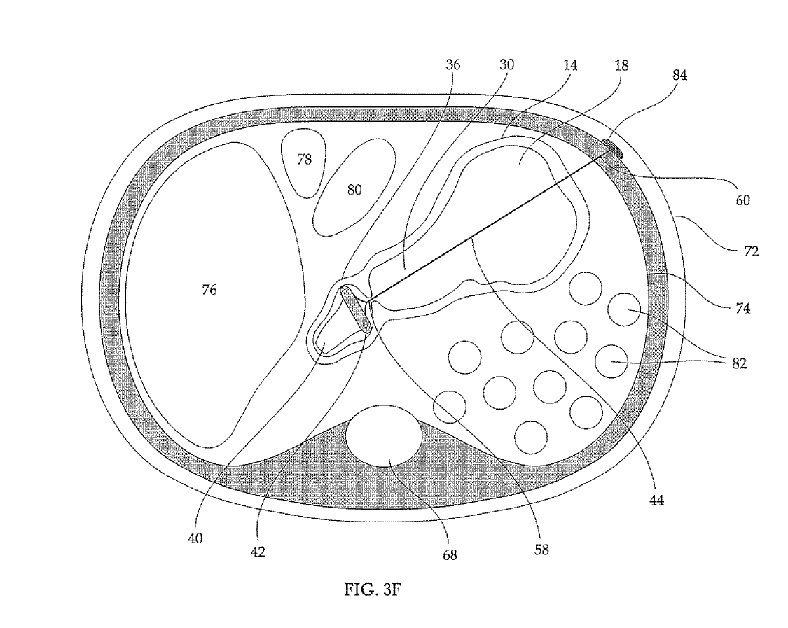

An embodiment of the method of the invention for deploying a duodenal liner device is described with reference to FIGS. 3A-3F, where a duodenal liner device such as duodenal liner device 38 described above with reference to FIGS. 2A-2C is deployed in the duodenum of a human.

In FIG. 3A, duodenal liner device 38 is depicted packed inside the bore of a 9 mm outer diameter substantially rigid tubular probe section 62 of a deployment device which includes a pusher 66 that slidingly fits inside the bore of probe section 62. Expandable centering component 42 is constrained to be in a collapsed configuration by the walls of probe section 62. Prior to being packed inside probe section 62 of a deployment device, distal end 52 of liner tube 40 is partially passed through proximal lumen opening 50 and twisted so as to block distal lumen opening 54. Proximal end 60 of tether 44 is tied to pusher 66 of the deployment device.

The deployment device is delivered sterilized and packaged in a sterility-preserving package, as is known in the art.

A subject needing deployment of a duodenal liner device is prepared. An area of the left side of the abdomen, roughly from the height of the top twelfth thoracic vertebra to the bottom of the second lumbar vertebra is anesthetized.

Under guidance of a medial imaging device (e.g., a standard ultrasound imaging device) a trocar is used to deploy a substantially rigid cannula having a 9 mm bore diameter to establish a transcutaneous channel from outside of the body of the subject through a gastric wall into a gastric cavity. As is seen in FIG. 3B (transverse cross section of a human at T12 68, from below), cannula 70 defines a substantially straight channel that passes through skin 72 and an abdominal muscle layer 74 from the left side to enter gastric cavity 18 of stomach 14 in line with the duodenal bulb 36, pyloric sphincter 32, pyloric antrum 30 and pyloric portion 28 of stomach 14 while avoiding liver 76, gall bladder 78, flexure of colon 80 and parts of small intestine 82.

In FIG. 3C, cannula 70 penetrates the gastric wall at the greater curvature 24 near pyloric portion 28 of stomach 14. Probe section 62 of the deployment device is directed through cannula 70, transporting duodenal liner device 38 into gastric cavity 18, through pyloric portion 28 and pyloric antrum 30 to pass through pylorus 34.

In FIG. 3D, pusher 66 of the deployment device is used to push duodenal liner device 38 out of probe section 62 into duodenal bulb 36. As discussed above, expandable centering component 42 is self-expanding. When released from the constraints of probe section 62, centering component 42 deploys in duodenal bulb 36, expanding from the collapsed configuration to the deployed configuration, dilating proximal lumen opening 50 of liner tube 40. Centering component 42 of duodenal liner device 38 is configured to expand to just fit or be somewhat smaller than duodenal bulb 36. As a result, centering component 42 lightly contacts walls of duodenal bulb 36 rather than tightly pressing against the walls of duodenal bulb 36.

In FIG. 3E, pusher 66 and probe section 62 of the deployment device are withdrawn. Withdrawal of pusher 66 withdraws proximal end 60 of tether 44 which trails after pusher 66, out of gastric cavity 18, out through the gastric wall and the channel defined by cannula 70, past abdominal muscle layer 74 and skin 72.

A distal end of a fluid conduit is passed through cannula 70 and into proximal lumen opening 50 that is dilated by centering component 42 (not depicted). A suitable fluid, such as isotonic saline solution, is passed through the fluid conduit to unwrap and deploy liner tube 40 inside the lumen of duodenum 16, substantially as described in U.S. Pat. No. 7,267,694.

Cannula 70 is withdrawn and the transcutaneous channel is allowed to close around tether 44. In FIG. 3F, proximal end of 60 of tether 44 is secured to a subcutaneous anchor 84 that comprises a pad that presses against the layer of abdominal muscle layer 74. Skin 72 is closed over anchor 84 and proximal end 60 of tether 44.

After a relatively short convalescence, the subject in which duodenal liner device 38 is implanted is ambulatory and can leave a substantially normal life. Duodenal liner device 38 functions substantially similarly to some duodenal liners known in the art and as described hereinabove.

When deployed, the length of liner tube 40, together with the peristaltic motion of duodenum 16, keeps duodenal liner device 38 from migrating proximally into the stomach. Since centering component 42 fits in duodenal bulb 36 which is somewhat wider than the descending portion of duodenum 16 together with the fact that that the descending portion curves sharply downward from duodenal bulb 36 means that centering component 42 tends to keep duodenal liner device 38 from migrating distally into the distal portions of the gastrointestinal tract. If, however, some failure occurs or if peristaltic motion is exceptionally great, tether 44 prevents duodenal liner device 38 from migrating distally.

Tether 44 which proximal end 60 is fixed to anchor 84, prevents duodenal liner device 38 from being drawn in a distal direction into duodenum 16 despite the fact that centering component 42 does not press tightly against the luminal walls of duodenal bulb 36. Since tether 44 is thin, pyloric sphincter 32 functions in a substantially usual way despite the fact that tether 44 passing through pylorus 34. Since centering component 42 does not press tightly against the luminal wall of duodenal bulb 36, centering component 42 does not substantially grow into the luminal walls, does not substantially distend duodenal bulb 36 and does not substantially influence the functioning or nature of the luminal surface of duodenal bulb 36.

Some embodiments of the present invention take advantage of a number of fortuitous anatomical details. As seen for, example in FIG. 3B, there is usually at least one straight path from duodenal bulb 36 that passes towards the left and slightly downwards, through pylorus 34, pyloric antrum 30, pyloric portion 28 and to an opposing gastric wall, the path continuing through the gastric wall through a layer of abdominal muscle layer 74, fat and emerging through skin 72 approximately at a height no higher than the top of the last thoracic vertebra (T12) and no lower than the bottom of the second lumbar vertebra (L2).

Since the position of duodenum 16 is relatively fixed in the body, the straight path through bodily tissue is relatively fixed, so there is little danger of cutting or tearing of tissue by a tether following the straight path. Thus, a person in which a duodenal liner is deployed in accordance with some embodiments of the invention can move and eat substantially normally, with little fear of damage from the tether.

A tether following a straight path passing through pylorus 34 from duodenal bulb 36 does not pass through or perforate sensitive organs such as intestines, allowing the tether to be safely transcutaneously deployed, maintained and removed, as detailed herein.

Further, a tether such as 44 following a straight path from duodenal bulb 36, through pylorus 34 and into pyloric antrum 30 does not substantially interfere with the functioning of pylorus 34, especially when the tether has a small diameter.

In some embodiments, such as when a tether follows a straight path as described above, the tether passes through a hole in gastric wall near the bottom of stomach 14, where gastric juices accumulate. Surprisingly, in some embodiments gastric juices do not substantially leak from the hole. Although not wishing to be held to any one theory, it is believed that the lack of substantial leakage is attributable to a combination of one or more factors including the small diameter of a typical tether and consequently small size of the hole, the seal formed by gastric tissue constricting around a tether and the ability of gastric tissue to quickly and effectively heal.

In the embodiment for deploying duodenal liner device 38 described above, tether 44 is functionally associated with proximal end 48 of liner tube 40 prior to deployment of expandable centering component 42 in duodenal bulb 36. In some embodiments, a centering component of a duodenal liner device, without a tether, is first deployed in a duodenal bulb and subsequently a tether is functionally associate with the other components of the duodenal liner device such as the centering component.

In the embodiment for deploying a duodenal liner device described above, tether 44 is transported through the channel defined by cannula 70 into gastric cavity 18 and then proximal end 60 of tether 44 is withdrawn back out of gastric cavity 18 through the channel using pusher 66. In some embodiments, the proximal end of a tether is maintained at the proximal end of the channel (e.g., outside of the body) while the distal end of the tether (in some cases already functionally associated with the centering component of the duodenal liner device) is transported through the channel and into the gastric cavity.

In the embodiment for deploying a duodenal liner device described above, liner tube 40 is unfolded to be deployed in duodenum 16 with a fluid forced in through proximal lumen opening 50. In some embodiments, other methods of unfolding are used. For example, in some embodiments a solid liner tube deployer is placed in a proximal lumen opening and used to unfold the liner tube, for example a liner tube deployer such as the guide ball described in Tarnoff M, Shikora S, Lembo A, Gersin K in Surg Endosc 2008, 22, 1023-1028.

In the embodiment for deploying a duodenal liner device described above, probe section 62 of the deployment device is substantially rigid. In some embodiments, a probe section of a device for deploying a duodenal liner device of the invention is relatively flexible.

In the embodiment for deploying a duodenal liner device described above, the channel in the body of the subject is first formed, supported using cannula 70 and then used for passage of probe section 62 of the deployment device to transport duodenal liner device 38 through the channel, into gastric cavity 18 and through pylorus 34. In some embodiments, a probe section of a deployment device is configured to form a channel while transporting a duodenal liner device.

After a time there may be a need to examine, maintain, replace or remove a deployed duodenal liner device.

An aspect of the present invention relates to a method for removing a duodenal liner deployed in the duodenum of a human subject, where the duodenal liner device is at least partially secured in place with a tether. In some embodiments, a method of removal takes advantage of the fact that the tether defines a transcutaneous channel to the duodenum, the duodenal bulb and the duodenal liner device deployed therein.

In some embodiments, the method of removing the duodenal liner device includes a) exposing the proximal end of the tether; b) guiding a probe section of an extraction device through a channel defined by the tether in the body of the subject to proximity of a proximal end of the duodenal liner device; c) placing at least a portion of the duodenal liner device inside the probe section of the extraction device; and d) extracting the probe section of the extraction device from the body of the subject through the channel, thereby removing the duodenal liner device.

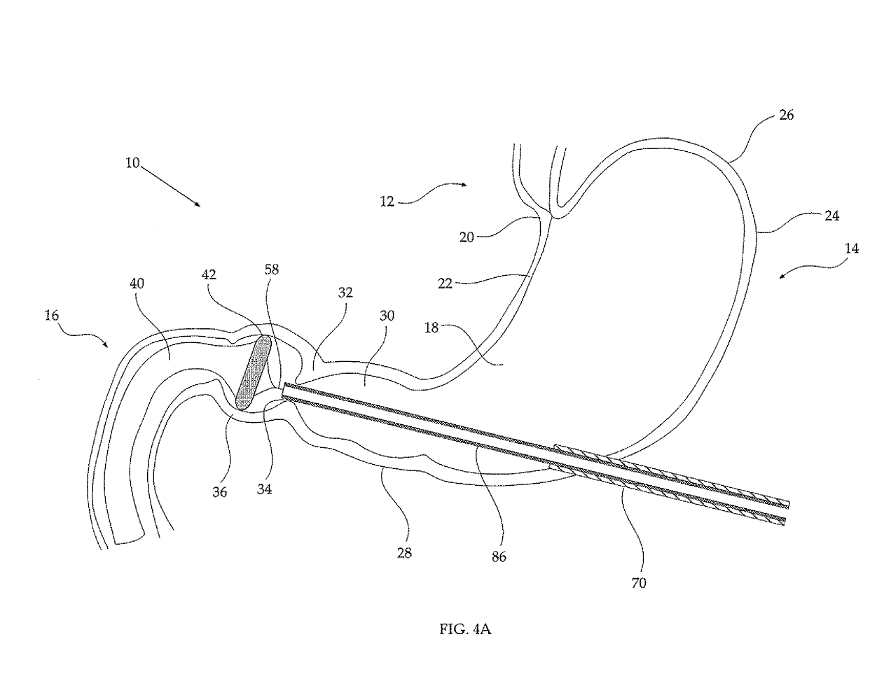

An embodiment of the method of the invention for removing a duodenal liner device is described with reference to FIGS. 4, where a duodenal liner device such as 38 described above with reference to FIGS. 2 and 3 is removed from the duodenum of a human.

Skin 72 is cut to expose anchor 84 and proximal end 60 of tether 44. Proximal end 60 of tether 44 is detached from anchor 84 and anchor 84 removed.

A balloon catheter is guided along tether 44 (tether 44 may be considered as functioning as a guide wire) through the channel in the body of the subject along with a rigid cannula 70, not depicted. When needed, the balloon catheter is inflated to push apart tissue and increase the radial diameter of the channel to accommodate cannula 70.

In FIG. 4A, an axially rigid tubular probe section 86 of an extraction device is advanced through cannula 70 into gastric cavity 18 and through pylorus 34 to proximity of proximal end 48 of liner tube 40 of duodenal liner device 38.

In FIG. 4B, while probe section 86 of the extraction device is held firmly in place, proximal end 60 of tether 44 is pulled, pulling the three strands at distal end 58 of tether 44 into probe section 86 of the extraction device, which apply a force that collapses expandable centering component 42 to a collapsed configuration and at least partially into the bore of probe section 86 of the extraction device.

Probe section 86 of the extraction device is carefully retracted through cannula 70, removing duodenal liner device 38 from duodenum 16.

In the embodiment for removing duodenal liner device 38 from a duodenum described above, the radial diameter of the channel through the body is increased prior to guiding of probe section 62 of the extraction device to proximity with the proximal end of duodenal liner device 38. In some embodiments, the probe section of the extraction device is configured to widen the channel while advancing. In some such embodiments, the probe section increases the radial diameter of the channel while being guided through the channel along the tether.

In the embodiment for removing a duodenal liner device described above, tether 44 is pulled while probe section 86 of the extraction device is held in place in order to pull centering component 42 of duodenal liner device 38 into probe section 86 of the extraction device. In some embodiments, a tether is held in place while a probe section of an extraction device is pushed in a distal direction over a centering component of a duodenal device.

In duodenal liner device 38 described above, expandable centering component 42 is a separate component, wire loop 56, encased inside a rolled proximal portion of liner tube 40. In some embodiments, an expandable centering component is integrally formed with a liner tube 40. For example, in some embodiments, a duodenal liner device of the invention comprises a liner tube of an elastomer, for example silicone rubber, that is rolled-over at a proximal end (in the manner of prophylactic condoms) to provide a self-expanding ring of material that is integrally formed with the liner tube.

In duodenal liner device 38 described above, tether 44 is functionally associated with proximal end 48 of liner tube 40 at three points around the perimeter of proximal end 48 and centering component 42. In some embodiments, a tether is functionally associated with a liner tube or a centering component at one or two points so as to reduce the chance of interference with the passage of chyme into the proximal lumen opening of the liner tube. In some embodiments, a tether is functionally associated with a liner tube or a centering component at more than three points.

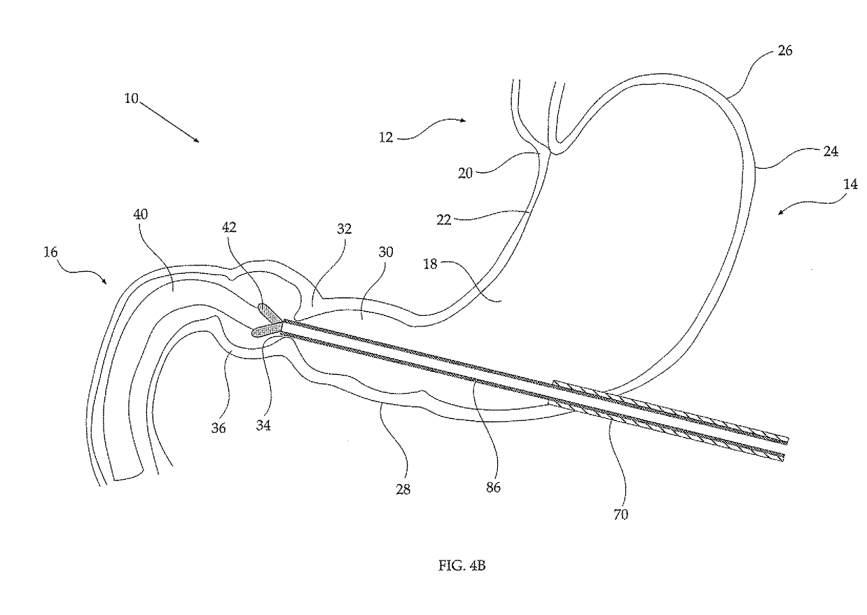

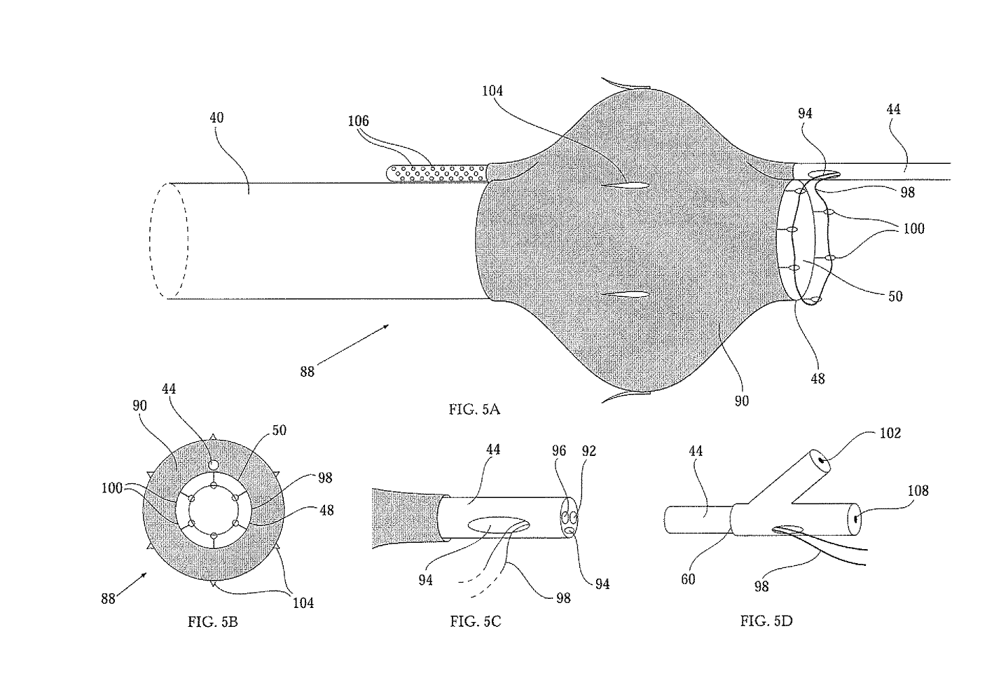

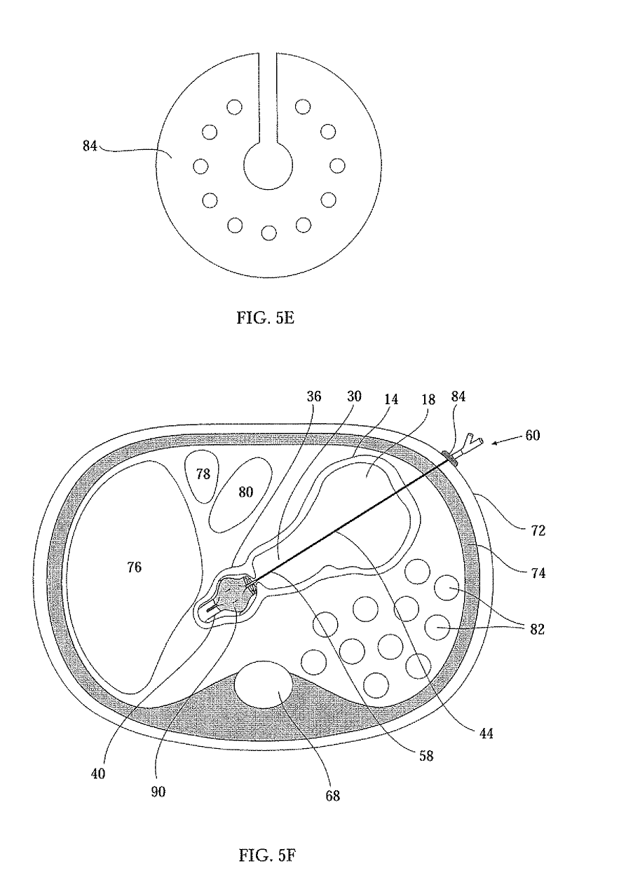

Additional embodiments of aspects of the invention are described with reference to FIGS. 5A-5F.

A duodenal liner device 88 is depicted in FIGS. 5. In FIG. 5A, duodenal liner device 88 is depicted in side view. In FIG. 5B, duodenal liner device 88 is viewed from the front. In FIG. 5C, a cross-section of tether 44 is depicted. In FIG. 5D, proximal end of tether 44 is depicted. In FIG. 5E, an extracutaneous anchor 84 is depicted. In FIG. 5F, duodenal liner device 88 is depicted deployed in a duodenum 16. Duodenal liner device 88 is similar to embodiments described above with a number of differences.

In duodenal liner device 88, the expandable centering component is a balloon 90 (e.g., of an elastomer such as silicon rubber, latex rubber or polyurethane such as used for fashioning a retention balloon of a commercial PEG device) which is welded over proximal end 48 of liner tube 40. Tether 44 is a silicon rubber or polyurethane tube with a 4 mm outer diameter and three lumens: balloon inflation lumen 92, drawstring lumen 94 and composition transport lumen 96 (see detail FIG. 5C)

Around the perimeter of proximal end 48 of liner tube 40 are six polyethylene eyelets 100. Drawstring 98, a polyethylene filament is looped through eyelets 100 and both ends of drawstring 98 pass together through drawstring lumen 94 to emerge from proximal end 60 of tether 44.

Balloon inflation lumen 92 is in fluid communication with and is thereby functionally associated with balloon 90. Balloon inflation lumen 92 serves as a conduit for transport of a fluid to controllably change the configuration of balloon 90. At the proximal end of balloon inflation lumen 92 is inflation port 102. Inflation port 102 comprises a valve ordinarily biased in a close position, preventing fluid from entering or exiting balloon inflation lumen 92. However, when an inflation device engages inflation port 102, the valve opens, allowing the inflation device to drive fluid into or out of balloon inflation lumen 92, leading to inflation or deflation of balloon 90.

Balloon 90 is reversibly expandable from a collapsed configuration with little or no fluid inside balloon 90 to a deployed configuration with a greater amount of fluid inside balloon 90, the fluid transported into and out of balloon 90 through balloon inflation lumen 92. In a deployed configuration depicted in FIGS. 5A and 5B, balloon 90 adopts a rounded-barrel shape having an axial length not less than a radius, roughly the shape of the internal cavity of a duodenal bulb 36.

Welded to the outer surface of balloon 90 around the greatest diameter in a deployed state of balloon 90 are six distally directed protrusions, barbs 104 (e.g., of polyurethane or stainless steel).

The tube which constitutes tether 44 continues through balloon 90 approximately 2 cm beyond the distal end of balloon 90. Starting immediately beyond the distal end of balloon 90, the walls of the tube are perforated with a plurality of nozzles 106, small holes that provide fluid communication between composition transport lumen 96 and the outside of the tube. At proximal end 60 of tether 44, composition transport lumen is in fluid communication with composition injection port 108. Composition injection port, composition transport lumen 96 and nozzles 106 constitute a composition-administration component of duodenal liner device 88 for administering a fluid composition as a spray to a gastrointestinal tract in which deployed.

In some embodiments, duodenal liner device 88 is deployed substantially as described above with reference to duodenal liner device 38, with a number of differences.

After a transcutaneous channel is established from outside the body of the subject through the gastric wall, duodenal liner device 88 is transported past pylorus 34 while balloon 90 is deflated in a collapsed configuration and while proximal end 60 of tether 44 remains trailing outside the body.

Duodenal liner device 88 is ejected from probe section 62 of deployment device into duodenal bulb 36. Subsequently, a probe section 62 of deployment device is withdrawn.

A balloon inflation device is functionally associated with inflation port 102. The balloon inflation device is activated, forcing an inflation fluid (e.g., saline) into balloon 90 through balloon inflation lumen 92 to bring balloon 90 into a deployed configuration.

The extent of inflation and the size of balloon 90 in the deployed configuration are in accordance to clinical considerations. In some embodiments, balloon 90 is inflated to make only weak contact with the luminal walls of duodenal bulb 36 in order to reduce the influence of balloon 90 on the walls at the expense of somewhat reduced anchoring of duodenal liner device 88 by balloon 90. In some embodiments, balloon 90 is inflated to a greater extent so that balloon 90 presses against the luminal walls of duodenal bulb 36. In such embodiments, balloon 90 presses against the luminal walls of duodenal bulb 36 to provide better anchoring, but may damaging or effect the functioning of the luminal walls.

The balloon inflation device is detached from inflation port 102, sealing balloon inflation lumen 92. An anchor 84 (FIG. 5E) is slipped over proximal end 60 of tether 44, thus engaging proximal end 60 of tether 44. Anchor 84 (similar in construction to the extracutaneous anchor depicted in U.S. Pat. No. 4,666,433) is pushed along tether 44 in a distal direction to press against skin 72. Tether 44 is pulled outwards in a proximal direction to a degree determined by medical considerations to maintain duodenal liner device 88 properly positioned (FIG. 5F).

After a relatively short convalescence, the subject in which duodenal liner device 88 is implanted is ambulatory and can leave a substantially normal life. Duodenal liner device 38 functions substantially similarly to some duodenal liners known in the art.

Duodenal liner device 88 is maintained in proper position and is not drawn into the small intestine due to the combined anchoring of balloon 90, barbs 104 and tether 44. For example, when duodenal liner device 88 is pulled by peristaltic motion in a distal direction into the small intestine, barbs 104 contact the duodenal intima and flare outwards, increasing the resistance of duodenal liner device 88 to migration in the distal direction.

Due to the regular motion of the body and the gastrointestinal tract, the tension applied to tether 44 changes so that anchor 84 applies intermittently changing pressure to the skin, preventing migration of duodenal liner device 88 in a distal direction.

In accordance with medical considerations, compositions, such as pharmaceutical compositions (e.g., compositions including an active pharmaceutical ingredient) are administered to the subject through the composition-administration component of duodenal liner device 88. For example, when a handling physician determines that it is necessary to administer a composition, a composition dispensing device is used to composition injection port 108 to force the composition (e.g., a sprayable fluid composition) into composition transport lumen 96. When a liquid composition is pumped into composition transport lumen 96 at a sufficiently high pressure, nozzles 106 open and the composition is sprayed at the luminal walls of the duodenum in which deployed, in accordance with the teachings of PCT publication WO 2008/104968. Due to the length of the tube defining composition transport lumen 96 and the location of nozzles 106, the composition is administered in the superior portion of the duodenum and/or in the ascending portion of the duodenum. When pressure is relieved, the tube contracts, sealing nozzles 106 and preventing the influx of fluids from the gastrointestinal tract into composition transport lumen 96.

In some embodiments, a composition dispensing device engages a composition injection port 108 only when it is desired to administer a composition. In some embodiments, an automated composition dispensing device is continuously associated with composition injection port 108 and administers composition according to a predetermined schedule or in accordance with detection of some event, in accordance with the teachings of PCT publication WO 2008/104968.

If necessary, removal of duodenal liner device 88 from a duodenum 16 is substantially similar to the described above with a few differences.

A composition administration device (if attached) is detached from composition injection port 108 and anchor 84 is detached from tether 44.

As described above, a balloon catheter is guided along tether 44 through the channel in the body of the subject along with a rigid cannula 70. The expandable balloon catheter is inflated as needed to push apart tissue and increase the radial diameter of the channel to accommodate cannula 70.

A balloon inflation device engages inflation port 102 and is activated to remove fluid from balloon 90 through balloon inflation lumen 92, bringing balloon 90 to a collapsed configuration.

While balloon 90 is held in place in duodenal bulb 36 with tether 44 (and in some embodiments, with a probe or other elongated rigid device) the ends of drawstring 98 are pulled out through drawstring lumen 94, bringing eyelets 100 together and thereby closing proximal lumen opening 50 and reducing the outer dimensions of the proximal end of duodenal liner device 88.

Subsequently, duodenal liner device 88 is pulled outwards in a proximal direction using tether 44, removing duodenal liner device 38 from duodenum 16 and through the bore of cannula 70 and out of the body of the subject.

As barbs 104 point in a distal direction, barbs 104 do not interfere with smooth removal of duodenal liner device 88.

In duodenal liner device 88, the centering component is balloon 90 having a rounded barrel shape that conforms to the profile of the duodenal bulb, allowing a snug fit in a duodenal bulb even when not pressing against the luminal walls of the duodenal bulb. In some embodiments, the centering component of a duodenal liner device is a balloon having a different shape, in embodiments, spiral, cylindrical, ring-shaped, partial ring-shaped or toroidal.

Duodenal liner device 88 is provided with distally directed features, barbs 104 attached to the outer surface of balloon 90, that are configured to increase the resistance of duodenal liner device 88 to migrate in a distal direction into the small intestine without substantially affecting the resistance to movement in a proximal direction. In some embodiments, other types of directional features are implemented to increase the resistance of duodenal liner device 88 to migrate in a distal direction. For example, in some embodiments, the outer surface of the centering component is textured or patterned for example with chevron or triangular shaped ridges pointing in a proximal direction. In some embodiments, a surface of a expandable centering component such as a balloon is smooth and does not include such features.

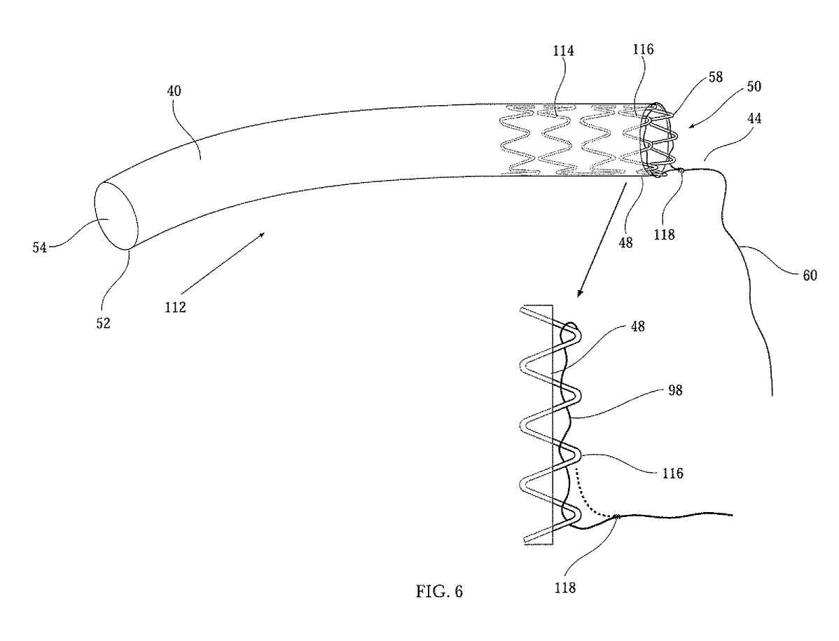

Additional embodiments of aspects of the invention are described with reference to FIG. 6 depicting duodenal liner device 112 in side view, together with an inset showing details of proximal end 48 of liner tube 40. Duodenal liner device 112 is similar to embodiments described above with a number of differences.

In duodenal liner device 112, balloon-expandable stent 114 functions as an expandable centering component. Stent 114 is a secured at proximal end 48 of liner tube 40 inside the lumen of liner tube 40 with sutures so that undulations 116 at the proximal end of stent 114 protrude from proximal lumen opening 50. Looped about the protruding end of stent 114 and weaving above and below the protruding undulations 116 is distal end 58 of tether 44 which contacts stent 114 and is thereby functionally associated with proximal end 48 of liner tube 40. Tether 44 is slidingly knotted about itself at knot 118, thus configured to act as a drawstring. If knot 118 is held while a proximal end 60 of tether 44 is pulled, looped distal end 58 of tether 44 constricts stent 114 to a collapsed configuration.

Deployment of duodenal liner device 112 may be performed according to any suitable method, including substantially analogously to the described above. Stent 114 is not-self expanding, but is expandable by application of an outward radial force, for example with a force applied by a balloon of a balloon catheter as known in the art of stenting.

In some embodiments, stent 114 is crimped over a balloon of a balloon catheter for deployment. During transporting of duodenal liner device 112 into the stomach and through pylorus, stent 114 is in a collapsed configuration, crimped over the balloon of the balloon catheter. When stent 114 is properly positioned, for example in a duodenal bulb 36, the balloon of the balloon catheter is inflated, applying an outwards force that expands stent 114 to adopt a deployed configuration. The extent of expansion is in accordance to clinical considerations, as discussed above with relation to duodenal liner device 88. Once stent 114 is in an expanded deployed configuration, deploying liner tube 40 and securing tether 44 is performed substantially as described above.

Removal of a deployed duodenal liner device such as 112 may be performed in any suitable way, including analogously to the discussed above. In some embodiments, collapsing stent 114 in order to place stent 114 inside a probe section of an extraction device is accomplished by pulling proximal end 60 of tether 44 while holding knot 118 and/or stent 114 in place, allowing tether 44 to function as a drawstring to collapse stent 114 to a collapsed configuration.

In duodenal liner device 112, the expandable centering component comprises an expandable stent 114. In some embodiments, an expandable centering component comprises a self-expanding stent.

A component of duodenal liner device 112, tether 44 is configured to function as a drawstring to collapse the centering component stent 114 to a collapsed configuration. In some embodiments, a drawstring for collapsing a centering component is not a part of the duodenal liner device, but is an additional component used only when required.

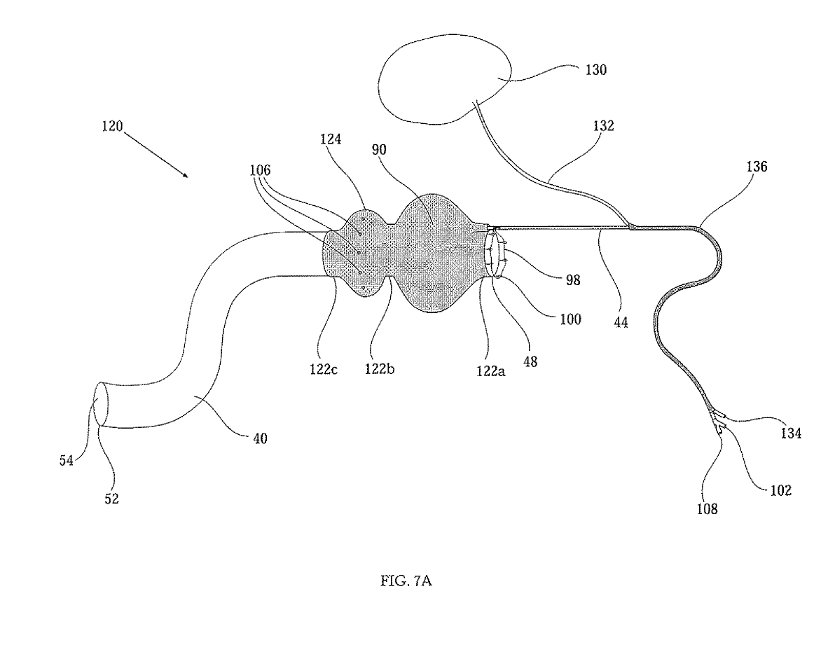

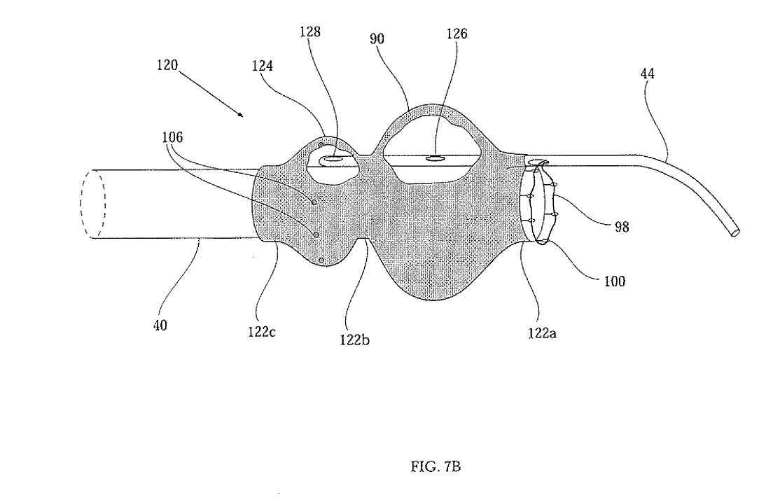

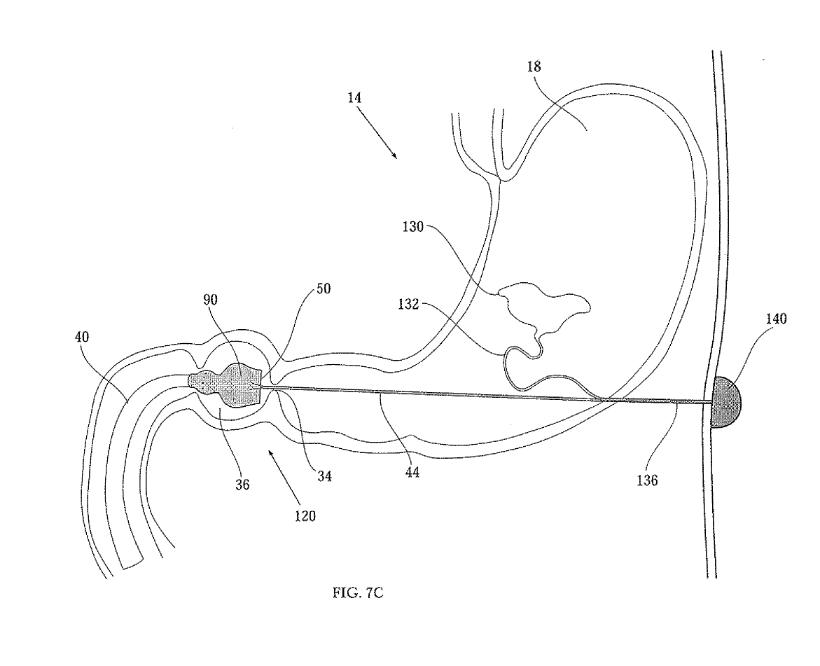

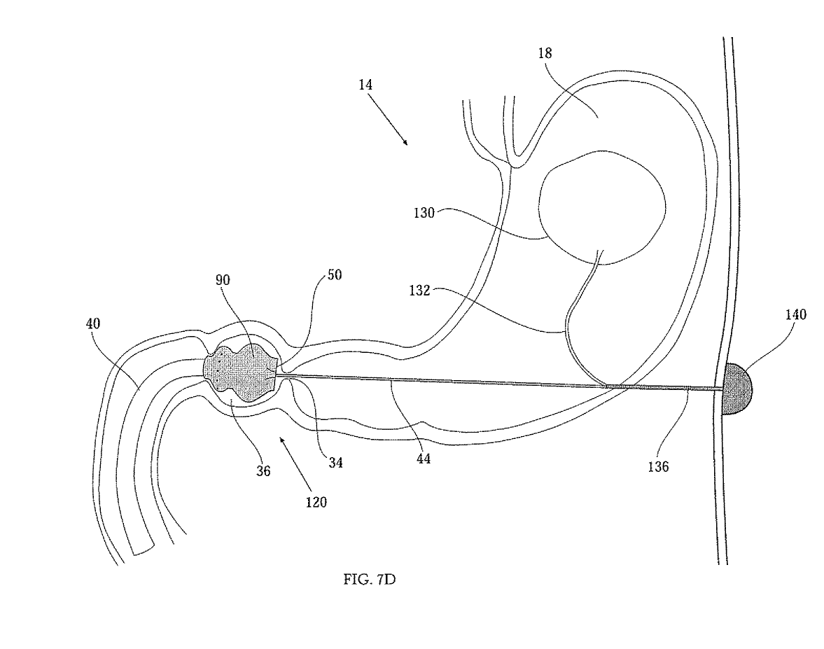

Additional embodiments of aspects of the invention are described with reference to FIGS. 7A-7C.

A duodenal liner device 120 is depicted in FIGS. 7. In FIG. 7A, duodenal liner device 120 is depicted in side view. In FIG. 7B, the proximal end of duodenal liner device 120 is depicted in side view with two “windows” showing internal details. In FIGS. 7C and 7D, duodenal liner device 120 is depicted in two states during use.

Duodenal liner device 120 is similar to other devices described above with a number of differences.

Around proximal end 50 of liner tube 40 (e.g., of polyurethane elastomer film) of duodenal liner device 120, a two-lobed tube of thin material (e.g., of polyurethane elastomer film) is secured with three circumferential welds 122a, 122b and 122c. Proximal weld 122a and middle weld 122b help define the expandable centering component of duodenal liner device 120, balloon 90. Middle weld 122b and distal weld 122c help define a composition dispensing chamber 124.

Duodenal liner device 120 includes eyelets 100 and a drawstring 98 similarly to duodenal liner device 88.

A tether 44 of duodenal liner device 120 is substantially similar to tether 44 of duodenal liner device 88 and also includes three lumens: balloon inflation lumen 92, drawstring lumen 94 and composition transport lumen 96, similar to the discussed above with reference to FIG. 5C. Similarly, the proximal end of tether 44 is substantially similar to the discussed above with reference to FIG. 5D and includes a balloon inflation port 102, a composition injection port 108 and a port through which the proximal ends of drawstring 98 emerge.

Similar to duodenal liner device 88, balloon inflation lumen 92 of duodenal liner device 120 is in fluid communication with and is functionally associated with balloon 90 through inflation hole 126 (FIG. 7B) so that balloon inflation lumen 92 serves as a conduit for transport of a fluid to controllably change the configuration of balloon 90.

Similar to duodenal liner device 88, the tube which constitutes tether 44 continues through balloon 90 and enters composition dispensing chamber 124. The tube has a composition dispensing port 128 in fluid communication with composition transport lumen 96. Small perforations (pinholes) through the walls of composition dispensing chamber 124 constitute nozzles 106.

Duodenal liner device 120 includes an inflatable gastric balloon 130 which is in fluid communication through gastric balloon inflation tube 132 with a gastric balloon inflation port 134. Composition injection port 108, composition transport lumen 96, composition dispensing chamber 124, composition dispensing port 128 and nozzles 106 constitute a composition-administration component of duodenal liner device 120 for administering a fluid composition to a gastrointestinal tract in which deployed.

Encasing part of the lengths of tether 44 and gastric balloon inflation tube 132 is a sleeve 136 of thin silicone rubber elastomer. Gastric balloon inflation port 134 and the proximal end of tether 44 emerge from a proximal end of sleeve 136. Gastric balloon inflation tube 132 and the distal end of tether 44 emerge from a distal end of sleeve 136.

Balloon 90 is reversibly expandable from a collapsed configuration with little or no fluid inside balloon 90 to a deployed configuration with a greater amount of fluid inside balloon 90, the fluid transported into and out of balloon 90 through balloon inflation lumen 92.

In some embodiments, duodenal liner device 120 is deployed substantially as described above with reference to duodenal liner device 88. A significant difference is that while liner tube 40 and balloon 90 are deployed in duodenum 16 and duodenal bulb 36, gastric balloon 130, in a deflated state, is deployed inside gastric cavity 18.

In some embodiments, a balloon inflation device is functionally associated with inflation port 102 and used, as described above with reference to duodenal liner device 88 to force an inflation fluid into balloon 90 through balloon inflation lumen 92 to bring balloon 90 into a deployed configuration. Subsequently, a balloon inflation device is functionally associated with gastric balloon inflation port 134 and used, to force an inflation fluid into inflatable gastric balloon 130 through gastric balloon inflation tube 132 to bring gastric balloon 130 into a deployed configuration to function as a standard volume-filling gastric balloon, as is known in the art.

In some embodiments, gastric balloon 130 is configured to be inflated and deflated, when deployed, automatically in response to a stimulus, automatically according to a predetermined schedule or manually according to the discretion of medical personnel. In some embodiments, the expandable centering component of duodenal liner device 120, balloon 90, is configured to be inflated and deflated, when deployed, automatically in response to a stimulus, automatically according to a predetermined schedule or manually according to the discretion of medical personnel.

Automatic and/or manual inflation or deflation of gastric balloon 130 and balloon 90 is substantially analogous to the dispensing of compositions in the gastrointestinal tract described in PCT patent publication WO 2008/104968 and to the changing of conformation of a duodenal obstructing device described in PCT patent publication WO 2008/096362, both of the Inventor which are both included by reference as if fully set forth herein. In some embodiments, automatic inflation and deflation of gastric balloon 130 and/or balloon 90 is performed in a manner analogous to the taught in U.S. Pat. No. 5,259,399 which is included by reference as if fully set forth herein. Analogous methods, sensors, event detectors, regimes and options described therein for triggering dispensing of a composition or for changing the conformation of a duodenal obstructor or for inflating/deflating a gastric balloon are implemented herein with the required changes for inflating and/or deflating gastric balloon 130 and/or balloon 90.

It is preferable that a subject in which duodenal liner device 120 is deployed be ambulatory. Thus, in some embodiments, a portable inflation pump is functionally associated with a gastric balloon inflation port allowing automatic inflation and deflation of a gastric balloon. Thus, in some embodiments, a portable inflation pump is functionally associated with an inflation port allowing automatic inflation and deflation of an expandable centering component. In duodenal liner device 120 depicted in FIGS. 7, portable inflation pump 140 is functionally associated with both inflation port 102 and with gastric balloon inflation port 134, and with sensors (not depicted) configured to detect gastrointestinal activity indicative of hunger.

After a relatively short convalescence, the subject in which duodenal liner device 120 is implanted is ambulatory and can leave a substantially normal life. Duodenal liner device 120 functions substantially similarly to the described above.

As described above with reference to duodenal liner device 88, a composition such as a pharmaceutical composition is optionally administered using duodenal liner device 120, in accordance with clinical considerations. A composition is forced through composition injection port 108 into composition transport lumen 96 to emerge through composition dispensing port 128 into composition dispensing chamber 124. When enough composition is forced therethrough, the walls of composition dispensing chamber 124 distend sufficiently to open nozzles 106 so that the composition is dispensed inside or near the duodenal bulb.

In some embodiments, both balloon 90 and gastric balloon 130 are usually in a substantially deflated state, FIG. 7C, so have little substantial effect on the gastrointestinal tract of the subject and cause little, if any discomfort Tether 44 prevents duodenal liner device 120 from being drawn into the small intestine

As depicted in FIG. 7D, when the sensors functionally associated with portable inflation pump 140 detect gastrointestinal activity associated with hunger, portable inflation pump 140 slowly forces fluid (ambient air) into balloon 90, centering and aligning proximal lumen opening 50 with pylorus 34 as well as applying pressure to mechanoreceptors in duodenal bulb 36, to induce a perception of satiety. Subsequently, portable inflation pump 140 slowly forces fluid (ambient air) into gastric balloon 130. Gastric balloon 130 contacts the walls of stomach 14, stimulating gastric mechanoreceptors to induce a perception of satiety in a manner analogous to gastric balloons known in the art. After a predetermined amount of time, portable inflation pump 140 is activated to deflate both balloons in a controlled fashion.

The extent of inflation and the size of balloon 90 and/or gastric balloon 130 at any moment in time are in accordance to clinical considerations, for example as described above with reference to duodenal liner device 88.

If necessary, removal of duodenal liner device 120 from a duodenum is substantially similar to the described above with reference to duodenal liner device 88.

In duodenal liner device 120, balloon 90 and gastric balloon 130 are inflated and deflated with ambient air, obviating the need for an inflation fluid reservoir functionally associated with portable inflation pump 140. In some embodiments, a portable inflation pump is functionally associated with an inflation fluid reservoir, and inflation fluid from the reservoir is forced into and pumped out of balloon 90 and/or gastric balloon 130 to inflate or deflate, respectively the balloons. Any suitable fluid can be used. For example, in some embodiments an inflation fluid reservoir contains saline or water.

In duodenal liner device 120, balloon 90 and gastric balloon 130 are both inflated by the same device, portable inflation pump 140. In some embodiments, each balloon has a dedicated portable inflation pump. In some embodiments, only one of the two balloons is associated with a portable inflation pump.

In duodenal liner device 120, when appropriate gastric activity is detected by the sensors, portable inflation pump 140 is activated to first inflate balloon 90 and subsequently to inflate gastric balloon 130. In embodiments, any suitable order of inflation of multiple balloons may be implemented in accordance with the invention. In some embodiments, portable inflation pump 140 is activated to inflate balloon 90 and gastric balloon 130 substantially simultaneously or concurrently. In some embodiments, portable inflation pump 140 is activated to first inflate gastric balloon 130 and subsequently to inflate balloon 90.

In embodiments of the invention discussed above, for example embodiments discussed with reference to FIGS. 3, liner tube 40 is twisted and folded to fit inside delivery probe section 62 (FIG. 3A). As discussed above, in such embodiments the distal end of delivery probe section 62 is advanced through pylorus 34, centering component 42 is deployed in duodenal bulb 36, and then liner tube 40 is straightened-out to be deployed inside duodenum 16, allowing passage of material such as chyme entering proximal lumen opening 50, through the lumen of line tube 40 and out through distal lumen opening 54.

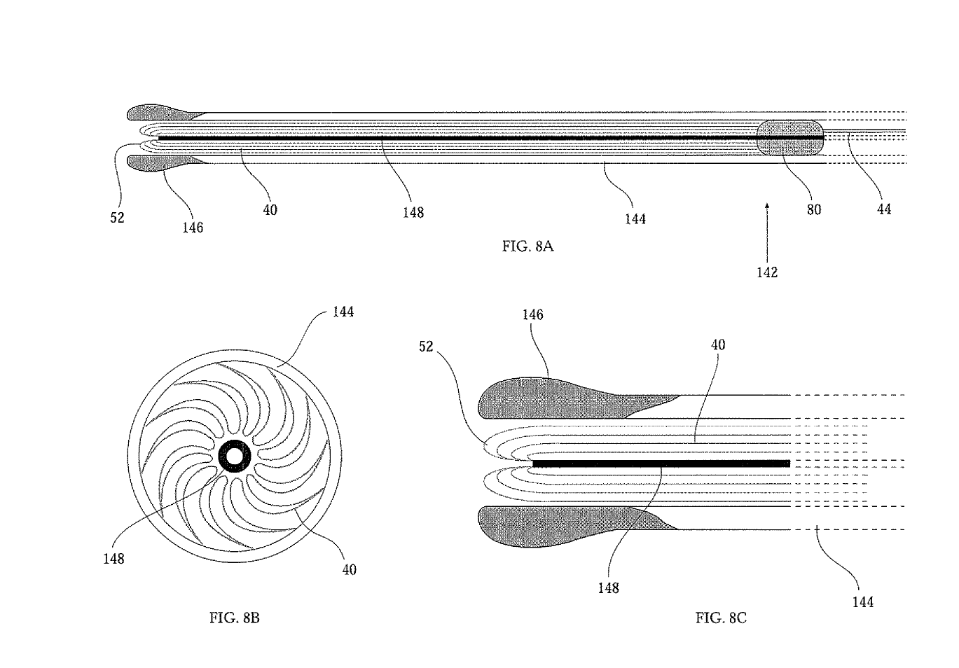

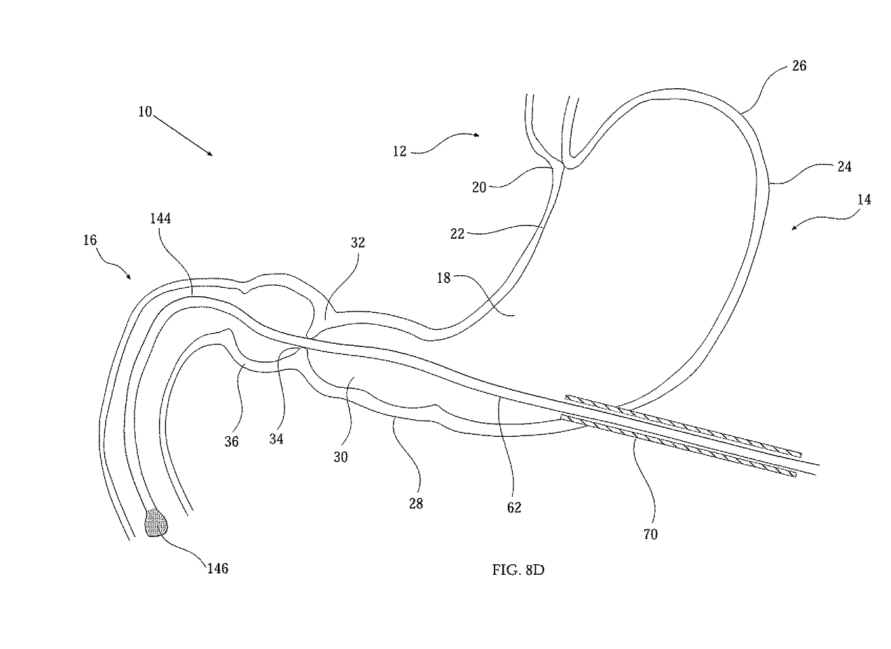

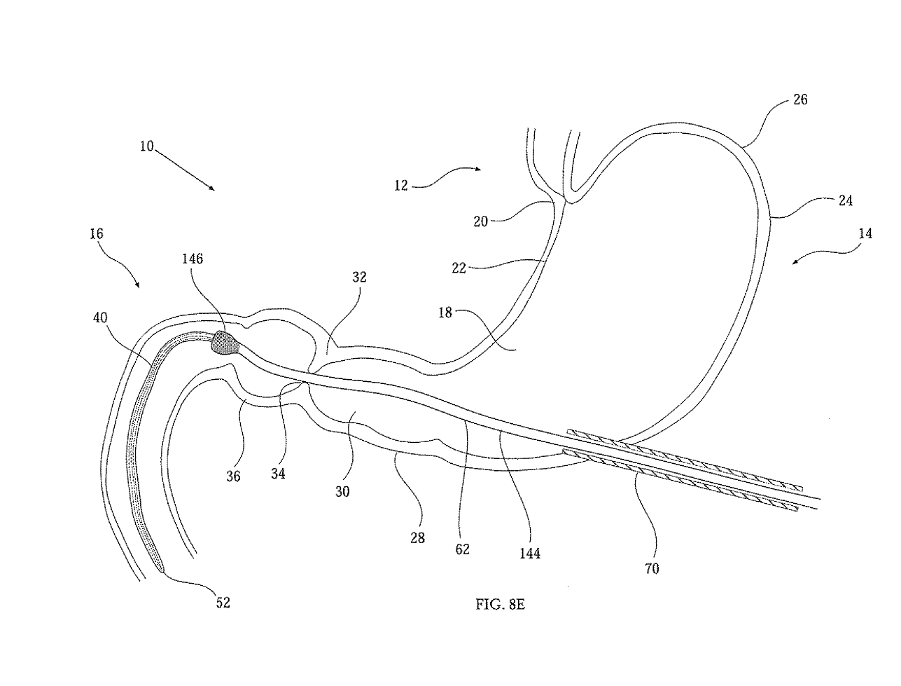

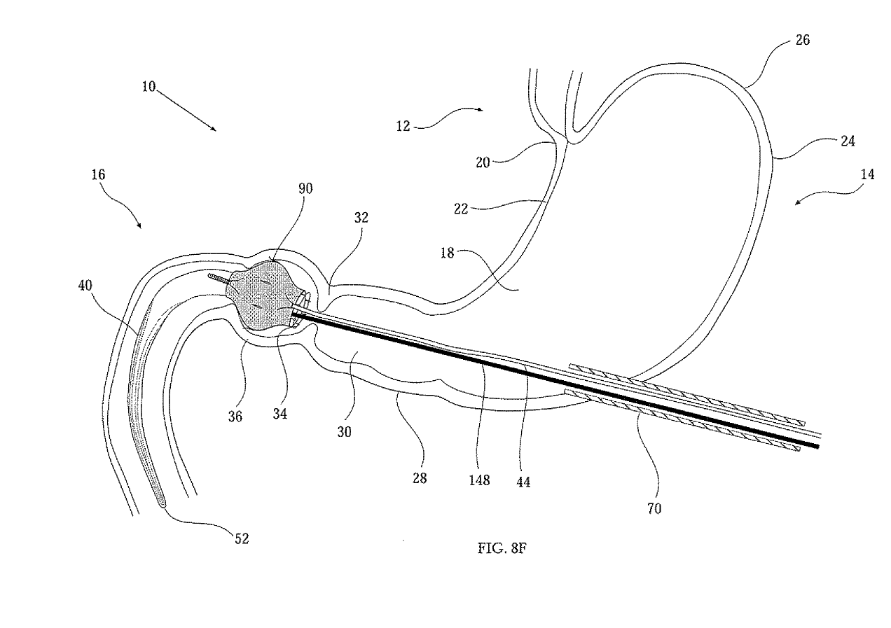

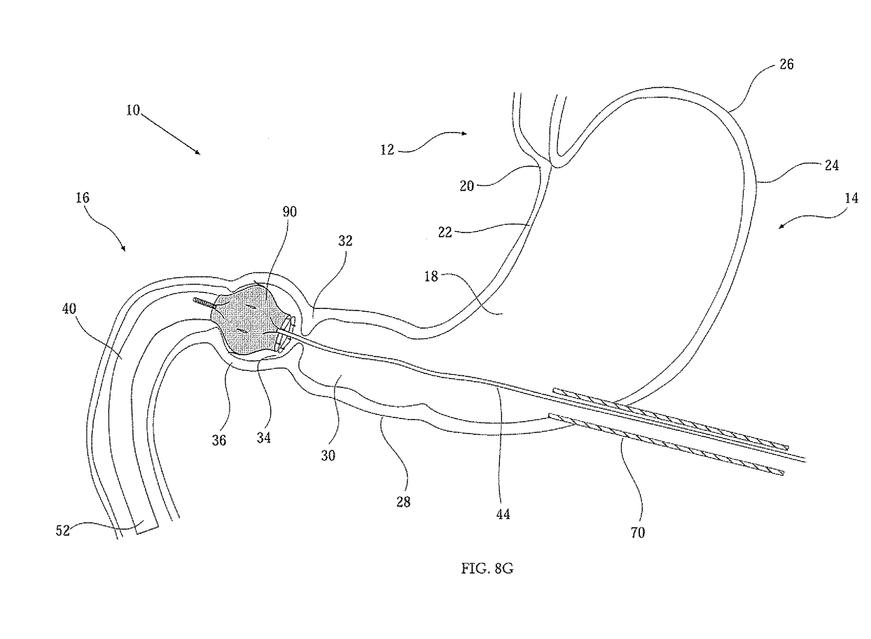

In some embodiments, a liner tube is not twisted or axially folded, but inwardly collapsed for delivery. In some such embodiments, the liner tube has a deployed length during the deployment process substantially similar to the deployed length of the liner tube. In some such embodiments, the duodenal liner device is deployed with the help of a flexible delivery probe that is configured to follow the curvature of the duodenum during delivery. One such embodiment, duodenal liner device 142 is depicted in FIG. 8.

Duodenal liner device 142 is substantially similar to duodenal liner device 88, discussed in detail with reference to FIGS. 5, but is deployed in a somewhat different fashion. In FIG. 8A, a liner tube 40 of duodenal liner device 142 packed inside a flexible delivery probe 144 of a delivery device is depicted in axial cross-section. In FIG. 8B, the axial cross section of a distal tip 146 of flexible delivery probe 144 is depicted in detail. In FIG. 8C, a radial cross section of liner tube 40 is depicted packed inside flexible delivery probe 144.

Flexible delivery probe 144 is a tube of flexible but rigid material such as PEEK in which liner tube 40 as well as the expandable centering component, balloon 90, is packed, FIG. 8A. Distal tip 146 of flexible delivery tube is of a soft and pliant material such as silicone rubber.

When packed inside flexible delivery probe 144, liner tube 40 is inwardly collapsed into a stellate shape to defining creases, where the lumen of liner tube 40 is unobstructed, FIG. 8B.

Through the lumen of liner tube 40 passes a flexible guide tube 148 of Nitinol. About 1 cm of distal end 52 of liner tube 40 is folded over and stuffed into the lumen of flexible guide tube 148, FIG. 8C.

For deployment, a transcutaneous cannula 70 opening into gastric cavity 18 is deployed, as described above. Distal tip 146 of flexible delivery probe 144 is carefully guided through cannula 70, through pylorus 34 and into duodenum 16. Flexible delivery probe 144 bends to follow the duodenal lumen, FIG. 8D.

When flexible delivery probe 144 has been pushed far enough (e.g., according to direct observation with a medical imaging modality such as an ultrasound imaging device) so that balloon 90 is more or less inside duodenal bulb, flexible delivery probe 144 is pulled in a proximal direction while guide tube 148 is held in place, preventing duodenal liner device 142 from being carried back with flexible delivery probe 144, FIG. 8E.

After flexible delivery probe 144 has been completely withdrawn so that balloon 90 is found in duodenal bulb 36, tether 44 is anchored and balloon 90 is inflated as described above, FIG. 8F

A rod (not depicted) is passed through the lumen of guide tube 148, pushing out distal end 52 of liner tube 40, thereby clearing the lumen of liner tube 40. The rod is withdrawn so that duodenal liner device 142 is deployed, FIG. 8G.

As discussed above, many different materials known in the art may be used to implement the teachings of the invention. One particularly useful material is elastomeric polyurethane. Methods useful for the manufacture of thin walled polyurethane articles such as the liner devices of the invention are well-known to one skilled in the art and are described, for example, in PCT patent publications WO/1995/005097 and WO/2003/103741 and in U.S. Pat. No. 5,679,423 and U.S. Pat. No. 6,523,540. For example, a tubular mandrel

In some embodiments, the teachings of the present invention are combined with or used together with other treatments. For example, in some embodiments, the volume of the stomach is reduced for example, by one or more techniques such as suturing, stapling, lap-band deployment and deployment of a separate gastric balloon.

Embodiments of the present invention have been described herein primarily with reference to treatment of living human subjects. It is understood, however, that embodiments of the present invention are performed for the veterinary treatment of a non-human mammal, especially a pig or other swine.

Embodiments of the present invention have been described herein primarily with reference to treatment of living subjects. It is understood that application of the present invention for training and educational purposes (as opposed to treating a condition) falls within the scope of the claims, whether on a living non-human subject or on a dead subject, whether on a simulated human body, a human cadaver or on a non-human body, whether on a digestive tract isolated (at least partially) from a body, or on a body.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the invention.

Section headings are used herein to ease understanding of the specification and should not be construed as necessarily limiting.

1-25. (canceled)

26. A device comprising:
a liner tube,
wherein the liner tube comprises walls of a flexible material defining a liner lumen, a proximal end defining a proximal lumen opening, and a distal end defining a distal lumen opening, and
wherein the liner tube is sufficiently designed so as to be capable of residing inside a duodenum of a human subject;
an expandable balloon component,
wherein the expandable balloon component is functionally associated with the proximal end of the liner tube; and
a tether,
wherein the tether comprises an elongated flexible material having one or more lumens, a proximal end, and a distal end,
wherein one lumen of the tether is in fluid communication with the expandable balloon component,
wherein the proximal end of the tether engages an anchor, and
wherein the distal end of the tether engages at least one of the liner tube or the expandable balloon component,
wherein the anchor is sufficiently designed so as to be securable to a surface of the human subject for securing the liner tube inside the duodenum.

27. The device of claim 26 wherein the anchor is a tether anchor.

28. The device of claim 26 wherein the distal end of the tether engages the liner tube.

29. The device of claim 26 wherein the distal end of the tether engages the liner tube at multiple locations.

30. The device of claim 26 wherein the distal end of the tether engages the expandable balloon component.

31. The device of claim 26 wherein the distal end of the tether engages the liner tube and the expandable balloon component.

32. The device of claim 26 wherein the proximal end of the tether passes through a gastric wall of the human subject, and the anchor is secured to an outer surface of the gastric wall.

33. The device of claim 26 wherein the proximal end of the tether passes through a gastric wall as well as other tissue of the human subject, and the anchor is secured to the surface of a muscle layer.

34. The device of claim 26 wherein the proximal end of the tether passes through a gastric wall as well as other tissue to emerge through skin of the human subject, and the anchor is secured to an outer surface of the skin

35. The device of claim 26 wherein the expandable balloon component is sufficiently designed so as to be deployable in the duodenal bulb to sufficiently pass chyme from a stomach of the human subject through the proximal lumen opening.

36. The device of claim 26 wherein one lumen of the tether is in fluid communication with a composition-administration component to facilitate administration of a composition in the duodenum.

37. The device of claim 37 wherein the composition-administration component is associated with the liner tube.

38. The device of claim 37 wherein the composition-administration component is associated with the expandable balloon component.

39. A device comprising:
a liner tube,
wherein the liner tube comprises walls of a flexible material defining a liner lumen, a proximal end defining a proximal lumen opening, and a distal end defining a distal lumen opening, and
wherein the liner tube is sufficiently designed so as to be capable of residing inside a duodenum of a human subject;
an expandable balloon component,
wherein the expandable balloon component is functionally associated with the proximal end of the liner tube,
wherein the expandable balloon component has a collapsed configuration and an expanded deployed configuration, and
wherein the expandable balloon component is sufficiently designed so as to be capable of residing completely in a duodenal bulb of the human subject;
a percutanous endoscopic gastrostomy tube,
wherein the percutanous endoscopic gastrostomy tube comprises a channel-defining component and a percutanous endoscopic gastrostomy anchor; and
a tether,
wherein the tether comprises an elongated flexible material having one or more lumens, a proximal end, and a distal end,
wherein one lumen of the tether is in fluid communication with the expandable balloon component,
wherein at least a portion of the tether passes through the channel-defining component of the percutanous endoscopic gastrostomy tube,
wherein the proximal end of the tether engages the percutanous endoscopic gastrostomy anchor, and
wherein the distal end of the tether engages at least one of the liner tube or the expandable balloon component,
wherein the percutanous endoscopic gastrostomy anchor is sufficiently designed so as to be securable to an outer skin surface of the human subject for securing the liner tube inside the duodenum.

40. The device of claim 39 wherein the distal end of the tether engages the liner tube.

41. The device of claim 39 wherein the distal end of the tether engages the liner tube at multiple locations.

42. The device of claim 39 wherein the distal end of the tether engages the expandable balloon component.

43. The device of claim 39 wherein the distal end of the tether engages the liner tube and the expandable balloon component.

44. The device of claim 39 wherein the proximal end of the tether passes through a gastric wall of the human subject, and the percutanous endoscopic gastrostomy anchor is secured to an outer surface of the gastric wall.

45. The device of claim 39 wherein the proximal end of the tether passes through a gastric wall as well as other tissue of the human subject, and the percutanous endoscopic gastrostomy anchor is secured to the surface of a muscle layer.

46. The device of claim 39 wherein the proximal end of the tether passes through a gastric wall as well as other tissue to emerge through skin of the human subject, and the percutanous endoscopic gastrostomy anchor is secured to an outer surface of the skin

47. The device of claim 39 wherein the expandable balloon component is sufficiently designed so as to be deployable in the duodenal bulb to sufficiently pass chyme from a stomach of the human subject through the proximal lumen opening.

48. The device of claim 39 wherein one lumen of the tether is in fluid communication with a composition-administration component to facilitate administration of a composition in the duodenum.

49. The device of claim 48 wherein the composition-administration component is associated with the liner tube.

50. The device of claim 48 wherein the composition-administration component is associated with the expandable balloon component.

51. A device comprising:
a liner tube,
wherein the liner tube comprises walls of a flexible material defining a liner lumen, a proximal end defining a proximal lumen opening, and a distal end defining a distal lumen opening, and
wherein the liner tube is sufficiently designed so as to be capable of residing inside a duodenum of a human subject; and
an expandable balloon component,
wherein the expandable balloon component is functionally associated with the proximal end of the liner tube,
wherein the expandable balloon component has a collapsed configuration and an expanded deployed configuration, and
wherein the expandable balloon component is sufficiently designed so as to be capable of residing completely in a duodenal bulb of the human subject,
wherein a smooth outer surface of the expandable balloon component is sufficiently designed so as to be deployable in the duodenal bulb so as to sufficiently contact luminal walls of the duodenal bulb to anchor the liner tube inside the duodenum.

52. The device of claim 51 further comprising a tether, wherein the distal end of the tether engages at least one of the liner tube or the expandable balloon component.

53. The device of claim 51 wherein the expandable balloon component is manufactured from a compliant material.

54. The device of claim 51 wherein the compliant material is polyurethane.

55. The device of claim 51 wherein the expandable balloon component is sufficiently designed so as to be deployable in the duodenal bulb to sufficiently pass chyme from a stomach of the human subject through the proximal lumen opening.
